(19) United States
(12) Patent Application Publication
(10) Pub. No.: US 2008/0193411 A1
(43) Pub. Date: Aug. 14, 2008

(54) Anti-viral Compounds

(75) Inventors: Martin Robert Leivers, San Francisco, CA (US); Franz Ulrich Schmitz, Mill Valley, CA (US); Christopher Don Roberts, Belmont, CA (US); Ali Dehghani Mohammad Abadi, Campbell, CA (US)

Correspondence Address: FOLEY & LARDNER LLP, 975 PAGE MILL ROAD, PALO ALTO, CA, 94304, US

(73) Assignee: Genelabs Technologies, Inc.

(21) Appl. No.: 11/943,535

(22) Filed: Nov. 20, 2007

Related U.S. Application Data
(60) Provisional application No. 60/860,614, filed on Nov. 21, 2006.

(51) Int. Cl. A61K 38/21; C07D 207/16; A61K 31/40; A61P 31/12; C07D 403/04; A61K 31/4184

(52) U.S. Cl. 424/85.4; 548/531; 514/423; 548/309.7; 514/394

(57) ABSTRACT

Disclosed are compounds, stereoisomers, tautomers, pharmaceutically acceptable salts, or prodrugs thereof of having Formula (I), their preparation, use, and compositions thereof for treating an infection mediated at least in part by a virus in the Flaviviridae family of viruses, wherein A, L¹, V, W, T, Z, R, Y¹, and p are as defined herein.

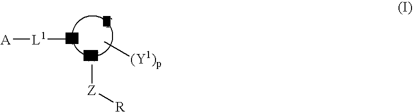

### CROSS-REFERENCE TO RELATED APPLICATIONS

This application claims the benefit under 35 U.S.C. §119(e) to U.S. provisional application Ser. No. 60/860,614 filed on Nov. 21, 2006, which is incorporated herein by reference in its entirety.

### BACKGROUND OF THE INVENTION

### Field of the Invention

The invention relates to the field of pharmaceutical chemistry, in particular to compounds, their preparation, compositions, and uses thereof for treating viral infections in patients mediated, at least in part, by a virus in the Flaviviridae family of viruses.

### REFERENCES

The following publications are cited in this application as superscript numbers:
1. Szabo, et al., Pathol. Oncol. Res. 2003, 9:215-221.
2. Hoofnagle J H, Hepatology 1997, 26:15 S-20S.
3. Thomson B J and Finch R G, Clin Microbial Infect. 2005, 11:86-94.
4. Moriishi K and Matsuura Y, Antivir. Chem. Chemother. 2003, 14:285-297.
5. Fried, et al. N. Engl. J. Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, et al., Ann. Rep. Med. Chem. 39, 223-237, 2004.
9. Watashi, et al, Molecular Cell, 19, 111-122, 2005
10. Horsmans, et al., Hepatology, 42, 724-731, 2005

All of the above publications are herein incorporated by reference in their entirety to the same extent as if each individual publication was specifically and individually indicated to be incorporated by reference in its entirety.

### STATE OF THE ART

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursuit to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4A protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

Notwithstanding the above, the discovery of new compounds active against one or more members of the Flaviviridae family of viruses would be beneficial particularly in view of the difficulty currently faced in treating diseases mediated, at least in part, by one or more of such viruses.

### SUMMARY OF THE INVENTION

This invention is directed to compounds, their preparation, compositions, prodrugs, and uses thereof for treating viral infections mediated, at least in part, by a virus in the Flaviviridae family of viruses. In one embodiment, provided is compound of Formula (I) or a stereoisomer, tautomer, pharmaceutically acceptable salt, or prodrug thereof, wherein:

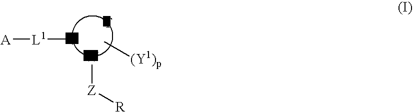

A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
L¹ is a bond, C₁-C₃ alkylene, C₁-C₂ heteroalkylene, C₂-C₃ alkenylene, or C₂-C₃ alkynylene;
T is C₂-C₆ alkylene or C₁-C₅ heteroalkylene and forms a 4-8 membered ring with V and W;
V and W are both CH, or one of V or W is CH and the other of V or W is N;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In one embodiment provided is a pharmaceutical composition comprising a pharmaceutically acceptable carrier and a therapeutically effective amount of a compound, stereoisomer, tautomer, pharmaceutically acceptable salt, or prodrug thereof of Formula (I).

In one embodiment provided is a method for treating a viral infection in a patient mediated at least in part by a virus in the Flaviviridae family of viruses, comprising administering to said patient a composition of Formula (I). In some aspects, the viral infection is mediated by hepatitis C virus.

### DETAILED DESCRIPTION

### Definitions

As used herein, the following definitions shall apply unless otherwise indicated.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy substitution is not attached to an acetylenic carbon atom.

“C₂-C₆ alkylene” refers to divalent straight chain alkyl groups having from 1 to 6 carbons.

“C₁-C₅ heteroalkylene” refers to alkylene groups where one or two —CH₂— groups are replaced with —S—, or —O— to give a heteroalkylene having one to five carbons provided that the heteroalkylene does not contain an —O—O—, —S—O—, or —S—S— group.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O)substituted alkyl, —NR⁴⁷C(O)cycloalkyl, —NR⁴⁷C(O)substituted cycloalkyl, —NR⁴⁷C(O)cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O)heterocyclic, and —NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR⁴⁷C(O)O-alkyl, —NR⁴⁷C(O)O-substituted alkyl, —NR⁴⁷C(O)O-alkenyl, —NR⁴⁷C(O)O-substituted alkenyl, —NR⁴⁷C(O)O-alkynyl, —NR⁴⁷C(O)O-substituted alkynyl, —NR⁴⁷C(O)O-aryl, —NR⁴⁷C(O)O-substituted aryl, —NR⁴⁷C(O)O-cycloalkyl, —NR⁴⁷C(O)O-substituted cycloalkyl, —NR⁴⁷C(O)O-cycloalkenyl, —NR⁴⁷C(O)O-substituted cycloalkenyl, —NR⁴⁷C(O)O-heteroaryl, —NR⁴⁷C(O)O-substituted heteroaryl, —NR⁴⁷C(O)O-heterocyclic, and —NR⁴⁷C(O)O-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring (e.g. fluorenyl). Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused, bridged, and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have only a single ring atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Metabolite” refers to any derivative produced in a subject after administration of a parent compound. The metabolite may be produced from the parent compound by various biochemical transformations in the subject such as, for example, oxidation, reduction, hydrolysis, or conjugation. Metabolites include, for example, oxides and demethylated derivatives.

“Prodrug” refers to art recognized modifications to one or more functional groups which functional groups are metabolized in vivo to provide a compound of this invention or an active metabolite thereof. Such functional groups are well known in the art including acyl groups for hydroxyl and/or amino substitution, esters of mono-, di- and tri-phosphates wherein one or more of the pendent hydroxyl groups have been converted to an alkoxy, a substituted alkoxy, an aryloxy or a substituted aryloxy group, and the like.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate [see Stahl and Wermuth, eds., “Handbook of Pharmaceutically Acceptable Salts”, (2002), Verlag Helvetica Chimica Acta, Zürich, Switzerland, for an extensive discussion of pharmaceutical salts, their selection, preparation, and use].

“Therapeutically effective amount” is an amount sufficient to treat a specified disorder or disease.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, the present invention provides a compound of Formula (I) or a stereoisomer, tautomer, pharmaceutically acceptable salt, or prodrug thereof, wherein:

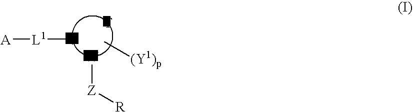

A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
L¹ is a bond, C₁-C₃ alkylene, C₁-C₂ heteroalkylene, C₂-C₃ alkenylene, or C₂-C₃ alkynylene;
T is C₂-C₆ alkylene or C₁-C₅ heteroalkylene and forms a 4-8 membered ring with V and W;
V and W are both CH, or one of V or W is CH and the other of V or W is N;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In one aspect, A is selected from the group consisting of

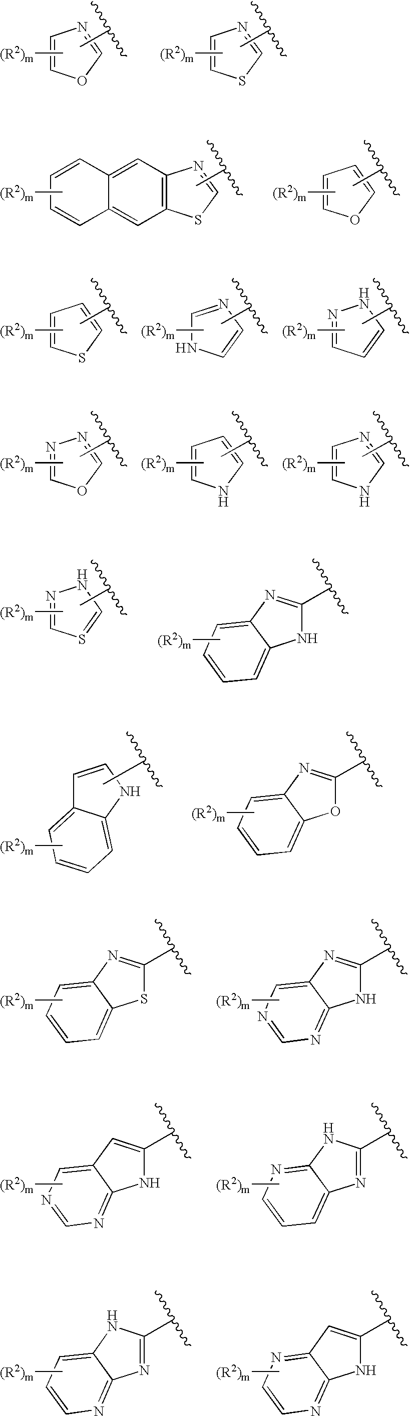

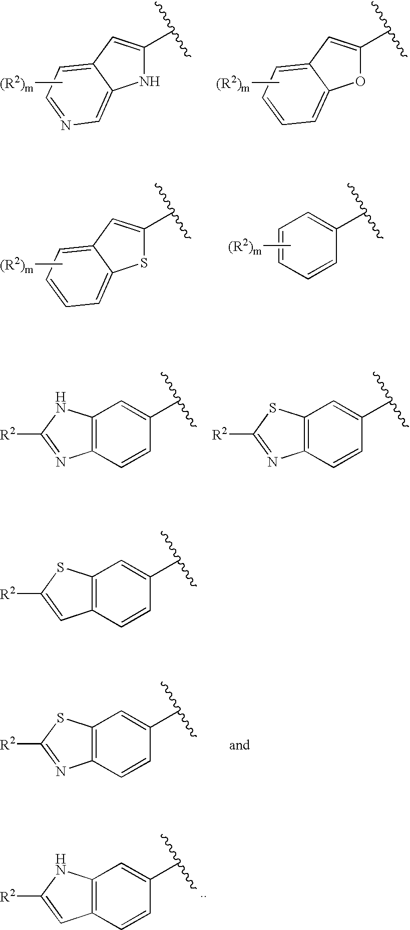

In one embodiment, provided is a compound of Formula (II) or a stereoisomer, tautomer, pharmaceutically acceptable salt, or prodrug thereof, wherein:

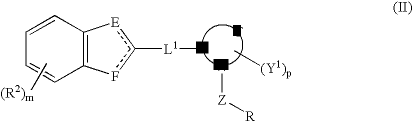

one of E or F is —N═ and the other of E or F is —S—, —O— or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
L¹ is a bond, C₁-C₃ alkylene, C₁-C₂ heteroalkylene, C₂-C₃ alkenylene, or C₂-C₃ alkynylene;
T is C₂-C₆ alkylene or C₁-C₅ heteroalkylene and forms a 4-8 membered ring with V and W;
V and W are both CH, or one of V or W is CH and the other of V or W is N;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In some aspects of the compound, stereoisomer, tautomer, pharmaceutically acceptable salt, or prodrug thereof of Formula (I) and (II), V is C and W is N.

In other aspects, T is —CH₂CH₂CH₂—.

In one embodiment, provided is a compound of Formula (III) or a stereoisomer, tautomer, pharmaceutically acceptable salt, or prodrug thereof, wherein:

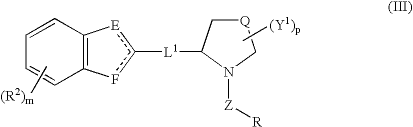

one of E or F is —N═ and the other of E or F is —S—, —O— or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
L¹ is a bond, C₁-C₃ alkylene, C₁-C₂ heteroalkylene, C₂-C₃ alkenylene, or C₂-C₃ alkynylene;
Q is selected from the group consisting of CH₂, CH(Y¹), C(Y¹)(Y¹), S, and O;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In some aspects of the compounds, stereoisomer, tautomer, pharmaceutically acceptable salt, or prodrug thereof of Formula (III), Q is S, CH₂, or O.

In some aspects of the compound, stereoisomer, tautomer, pharmaceutically acceptable salt, or prodrug thereof of Formula (I), (II), or (III), L¹ is a bond.

In other aspects, L¹ is C₂ alkynylene.

In some aspects at least one of R² is R³-L- wherein R³ is selected from the group consisting of aryl, substituted aryl, heteroaryl, substituted heteroaryl, heterocyclic, and substituted heterocyclic; and L, defined in the R³-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R³ is heterocyclic or substituted heterocyclic. In some aspects, L is a bond.

In some aspects, R³ is substituted phenyl. In some such aspects, said phenyl is substituted with one to three groups independently selected from alkyl, substituted alkyl, alkenyl, substituted alkenyl, alkoxy, substituted alkoxy, aryloxy, substituted aryloxy, alkylthio, substituted alkyl thio, acyl, acylamino, acyloxy, amino, substituted amino, aminocarbonyl, aminocarbonylamino, aminocarbonyloxy, aryl, substituted aryl, carboxyl, carboxyl ester, cyano cycloalkyl, substituted cycloalkyl, halo, hydroxy, heteroaryl, substituted heteroaryl, heterocyclic, substituted heterocyclic, nitro, thiol, alkylthio, and substituted alkylthio. In some aspects, at least one substitutent is cycloalkyl-C(O)NH—.

In some aspects, R³ is phenyl substituted with at least one group substituent selected from cyclopropyl-C(O)NH—, phenyl-C(O)NH—, cyclopentyl-C(O)NH—, 4-chlorophenyl-C(O)NH—, 4-chlorophenyl-C(O)NH—, methyl-C(O)NH—, methylamino, 4-methylphenyl-SO₂NH—, amino, ethyl-C(O)NH—, bromo, methoxy, methyl-SO₂NH—, chloro, phenyl-SO₂NH—, methyl-C(O)NH—, methyl-C(O)—, fluoro, methyl, ethyl, propyl, 4-fluorophenyl, nitro, phenyl, 4-bromobenzyloxy, cyclohexyl, isopropyl, tert-butyl, 4-methylpentyloxymethyl, NH₂C(O)—, hydroxy, cyclohexyl-NHC(O)CH₂S—, allyl, ethoxycarbonylmethylthio, dimethylamino, 3-nitro-phenyl, isobutyl, propoxy, butoxymethyl, butyl-C(O)NH—, methyl-NHC(O)—, ethyl-NHC(O)—, (2-oxo-hexahydro-thieno[3,4-d]imidazol-4-yl)-butyl-C(O)NH—, cyclopropyl-NHC(O)—, cyclohexyl-NHC(O)—, cyclopentyl-NHC(O)—, propyl, isobutyl, carboxy, pentyl-C(O)NH—, phenylamino-C(O)—, cylopropylamino-C(O)—, isopropylamino-C(O)—, and ethylamino-C(O)—.

In some aspects, Z is C(O).

In other aspects, R is OCH₂R¹ and R¹ is phenyl or substituted phenyl.

In other aspects, p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

In other aspects Y¹ is phenyl, pyridyl, substituted phenyl, or substituted pyridyl.

In other aspects Y¹ is pyridin-2-yl, pyridin-3-yl, pyridin-4-yl, 3-fluoro-pyridin-4-yl-, 2-hydroxy-pyridin-4-yl, tetrahydro-pyran-4-ylmethyl, phenyl, 2-fluoro-phenyl, 3-fluoro-phenyl, 4-fluorophenyl, 3-carboxy-phenyl, 4-carboxy-phenyl, 3-methoxycarbonyl-phenyl, 4-methoxycarbonyl-phenyl, 2-methoxy-phenyl, 3-methoxy-phenyl, 4-methoxy-phenyl, phenylmethyl, quinolin-4-yl, thiazol-2-yl, 3-cyano-phenyl, 4-cyano-phenyl, piperidin-3-yl-, piperidin-4-yl, pyrimidin-5-yl-, tetrahydro-pyran-4-yl, 2-chloro-pyridin-4-yl, cyclohexyl, oxazol-5-yl, 4-morpholin-4-ylmethyl-phenyl, 1-methyl-1H-imidazol-2-yl, or oxazol-2-yl.

In yet other embodiments, the present invention provides a compound, stereoisomer, tautomer, or a pharmaceutically acceptable salt thereof selected from Table 1.

TABLE 1

| Compound | Structure | Name |
|---|---|---|
| 4001 | 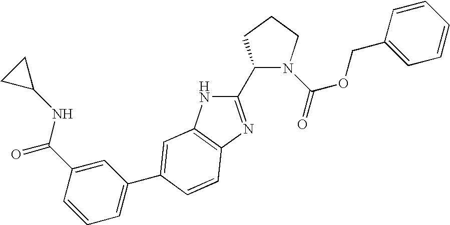 | 2-[6-(3-Cyclopropylcarbamoyl-phenyl)-1H-benzoimidazol-2-yl]-pyrrolidine-1-carboxylicacid benzyl ester |
| 4002 | 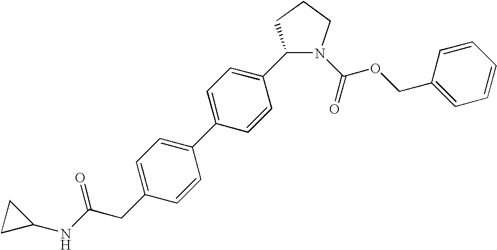 | 2-(4′-Cyclopropylcarbamoyl-methyl-biphenyl-4-yl)-pyrrolidine-1-carboxylicacid benzyl ester |
| 4003 | 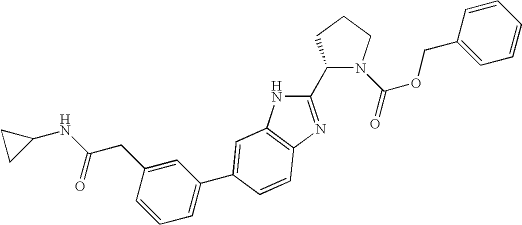 | 2-[6-(3-Cyclopropylcarbamoyl-methyl-phenyl)-1H-benzoimidazol-2-yl]-pyrrolidine-1-carboxylicacid benzyl ester |
| 4004 | 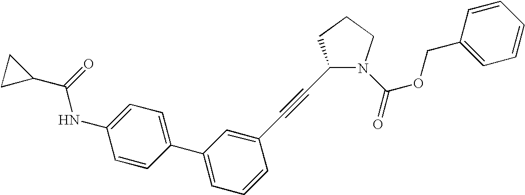 | 2-[4′-(Cyclopropanecarbonyl-amino)-biphenyl-3-ylethynyl]-pyrrolidine-1-carboxylic acid benzylester |
| 4005 | 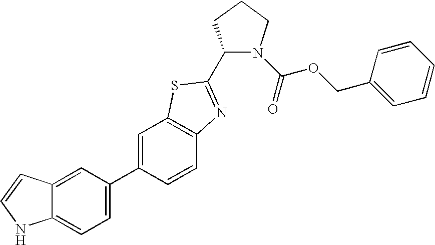 | 2-[6-(1H-Indol-5-yl)-benzothiazol-2-yl]-pyrrolidine-1-carboxylicacid benzyl ester |
| 4006 | 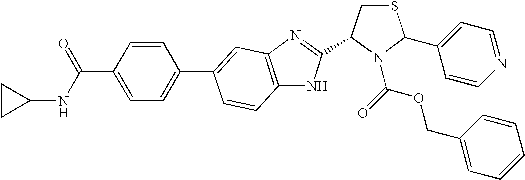 | 4-[5-(4-Cyclopropylcarbamoyl-phenyl)-1H-benzoimidazol-2-yl]-2-pyridin-4-yl-thiazolidine-3-carboxylic acidbenzyl ester |
| 4007 | 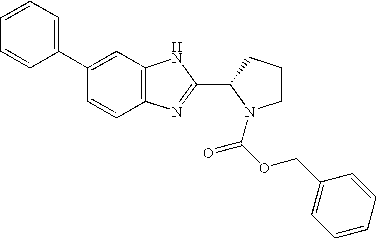 | 2-(6-Phenyl-1H-benzoimidazol-2-yl)-pyrrolidine-1-carboxylicacid benzyl ester |
| 4008 | 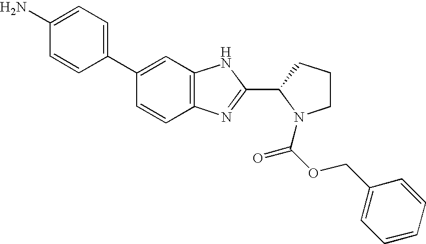 | 2-[6-(4-Amino-phenyl)-1H-benzoimidazol-2-yl]-pyrrolidine-1-carboxylicacid benzyl ester |
| 4009 | 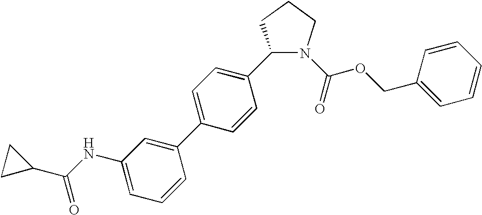 | 2-[3′-(Cyclopropanecarbonyl-amino)-biphenyl-4-yl]-pyrrolidine-1-carboxylicacid benzyl ester |
| 4010 | 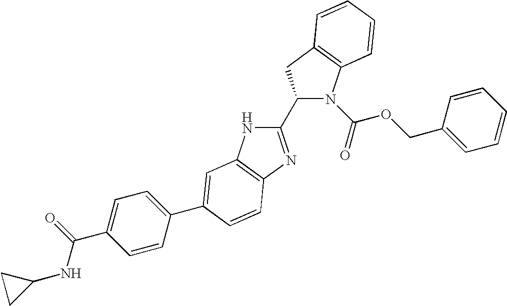 | 2-[6-(4-Cyclopropylcarbamoyl-phenyl)-1H-benzoimidazol-2-yl]-2,3-dihydro-indole-1-carboxylic acid benzylester |
| 4011 | 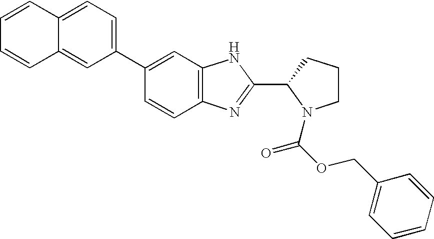 | 2-(6-Naphthalen-2-yl-1H-benzoimidazol-2-yl)-pyrrolidine-1-carboxylicacid benzyl ester |
| 4012 | 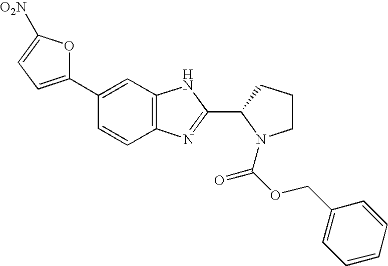 | 2-[6-(5-nitro-furan-2yl)-1H-benzoimidazol-2-yl)-pyrrolidine-1-carboxylicacid benzyl ester |
| 4013 | 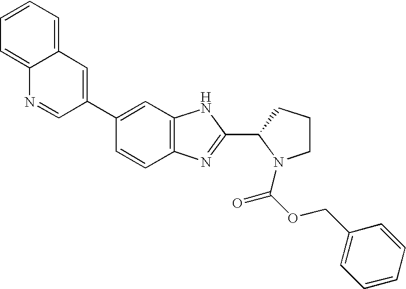 | 2-(6-Quinolin-3-yl-1H-benzoimidazol-2-yl)-pyrrolidine-1-carboxylicacid benzyl ester |
| 4014 | 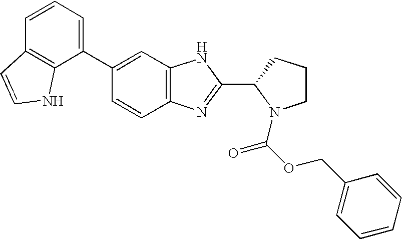 | 2-[6-(1H-Indol-7-yl)-1H-benzoimidazol-2-yl]-pyrrolidine-1-carboxylicacid benzyl ester |
| 4015 | 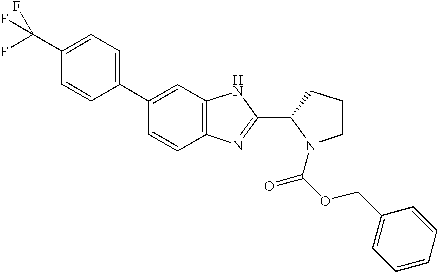 | 2-[6-(4-Trifluoromethyl-phenyl)-1H-benzoimidazol-2-yl]-pyrrolidine-1-carboxylicacid benzyl ester |
| 4016 | 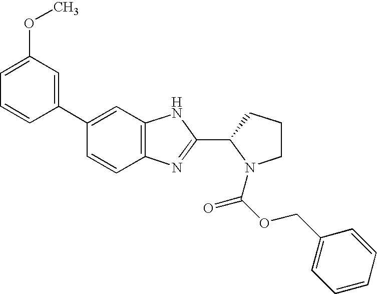 | 2-[6-(3-Methoxy-phenyl)-1H-benzoimidazol-2-yl]-pyrrolidine-1-carboxylicacid benzyl ester |
| 4017 | 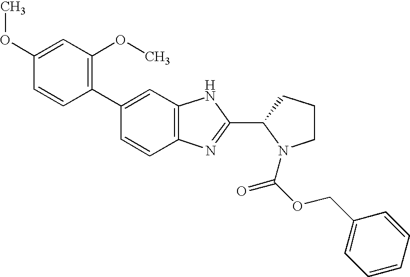 | 2-[6-(2,4-Dimethoxy-phenyl)-1H-benzoimidazol-2-yl]-pyrrolidine-1-carboxylicacid benzyl ester |
| 4018 | 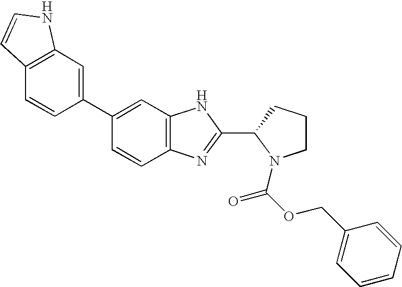 | 2-[6-(1H-Indol-6-yl)-1H-benzoimidazol-2-yl]-pyrrolidine-1-carboxylicacid benzyl ester |
| 4019 | 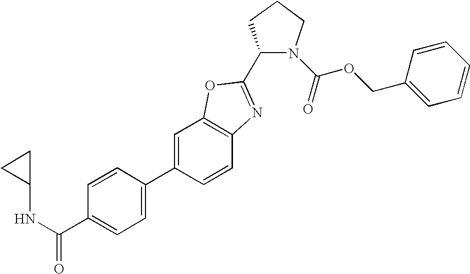 | 2-[6-(4-Cyclopropylcarbamoyl-phenyl)-benzooxazol-2-yl]-pyrrolidine-1-carboxylic acid benzylester |
| 4020 | 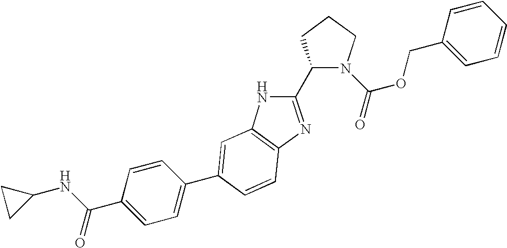 | 2-[6-(4-Cyclopropylcarbamoyl-phenyl)-1H-benzoimidazol-2-yl]-pyrrolidine-1-carboxylicacid benzyl ester |
| 4021 | 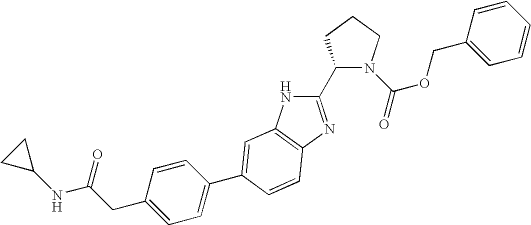 | 2-[6-(4-Cyclopropylcarbamoyl-methyl-phenyl)-1H-benzoimidazol-2-yl]-pyrrolidine-1-carboxylicacid benzyl ester |
| 4022 | 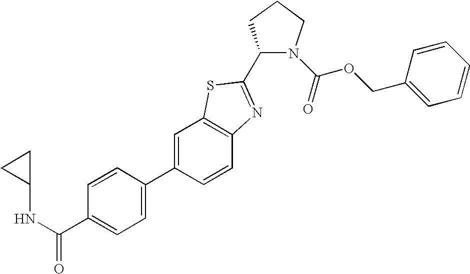 | 2-[6-(4-Cyclopropylcarbamoyl-phenyl)-benzothiazol-2-yl]-pyrrolidine-1-carboxylic acid benzylester |
| 4023 | 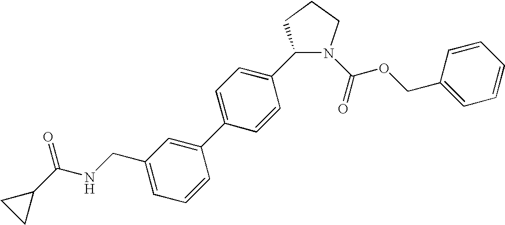 | 2-{3′-[(Cyclopropanecarbonyl-amino)-methyl]-biphenyl-4-yl}-pyrrolidine-1-carboxylic acid benzylester |
| 4024 | 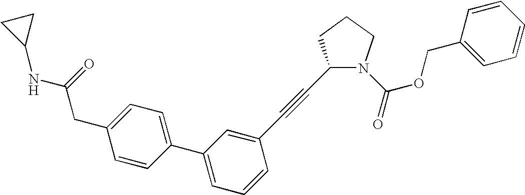 | 2-(4′-Cyclopropylcarbamoyl-methyl-biphenyl-3-ylethynyl)-pyrrolidine-1-carboxylic acid benzylester |
| 4025 | 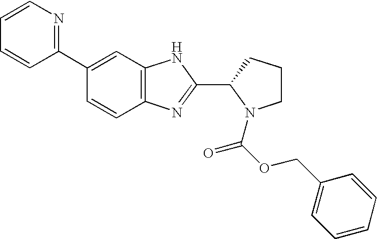 | 2-(6-Pyridin-2-yl-1H-benzoimidazol-2-yl)-pyrrolidine-1-carboxylicacid benzyl ester |
| 4026 | 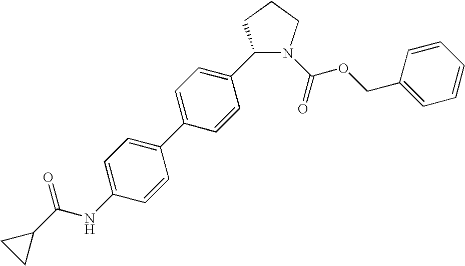 | 2-[4′-(Cyclopropanecarbonyl-amino)-biphenyl-4-yl]-pyrrolidine-1-carboxylicacid benzyl ester |
| 4027 | 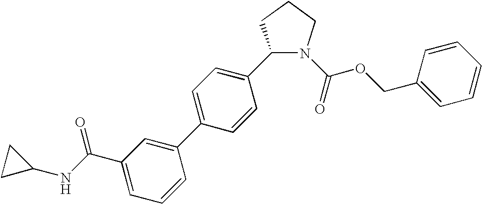 | 2-(3′-Cyclopropylcarbamoyl-biphenyl-4-yl)-pyrrolidine-1-carboxylicacid benzyl ester |
| 4028 | 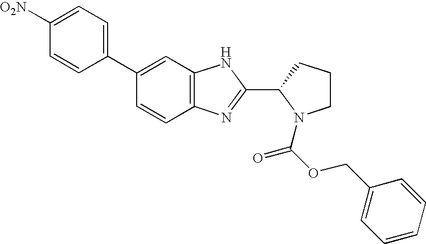 | 2-[6-(4-Nitro-phenyl)-1H-benzoimidazol-2-yl]-pyrrolidine-1-carboxylicacid benzyl ester |
| 4029 | 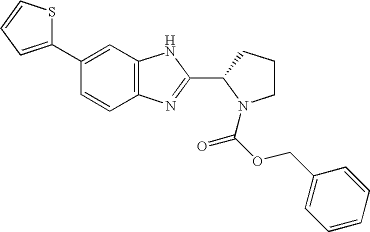 | 2-(6-Thiophen-2-yl-1H-benzoimidazol-2-yl)-pyrrolidine-1-carboxylicacid benzyl ester |
| 4030 | 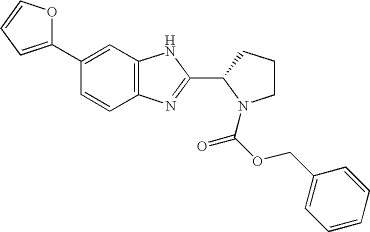 | 2-(6-Furan-2-yl-1H-benzoimidazol-2-yl)-pyrrolidine-1-carboxylicacid benzyl ester |
| 4031 | 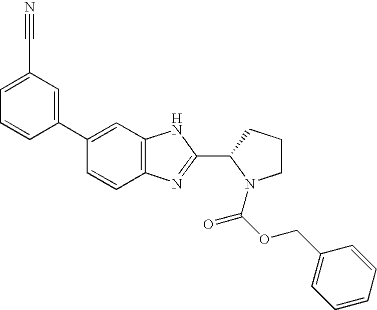 | 2-[6-(3-Cyano-phenyl)-1H-benzoimidazol-2-yl]-pyrrolidine-1-carboxylicacid benzyl ester |
| 4032 | 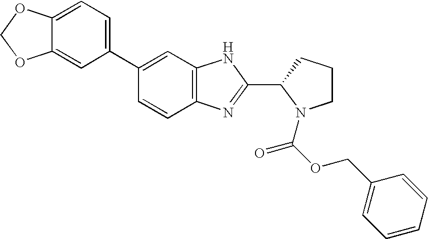 | 2-(6-Benzo[1,3]dioxol-5-yl-1H-benzoimidazol-2-yl)-pyrrolidine-1-carboxylic acid benzylester |
| 4033 | 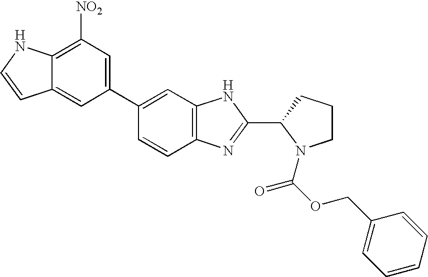 | 2-[6-(7-Nitro-1H-indol-5-yl)-1H-benzoimidazol-2-yl]-pyrrolidine-1-carboxylic acid benzylester |
| 4034 | 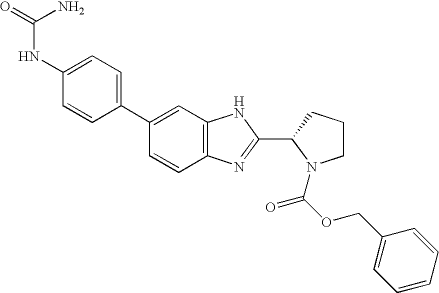 | 2-[6-(4-Ureido-phenyl)-1H-benzoimidazol-2-yl]-pyrrolidine-1-carboxylicacid benzyl ester |
| 4035 | 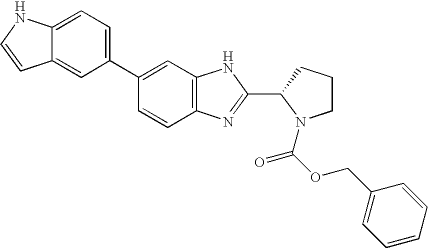 | 2-[6-(1H-Indol-5-yl)-1H-benzoimidazol-2-yl]-pyrrolidine-1-carboxylicacid benzyl ester |
| 4036 | 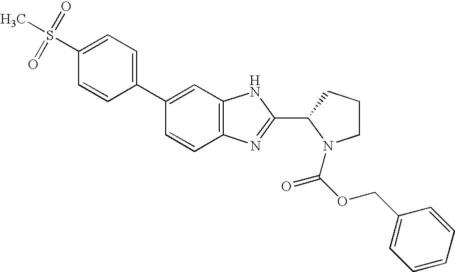 | 2-[6-(4-Methanesulfonyl-phenyl)-1H-benzoimidazol-2-yl]-pyrrolidine-1-carboxylicacid benzyl ester |
| 4037 | 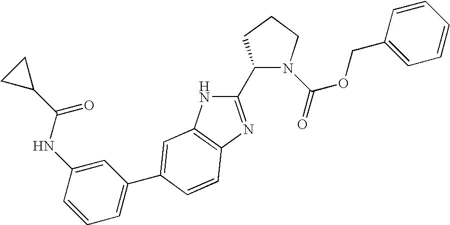 | 2-{6-[3-(Cyclopropanecarbonyl-amino)-phenyl]-1H-benzoimidazol-2-yl}-pyrrolidine-1-carboxylicacid benzylester |
| 4038 | 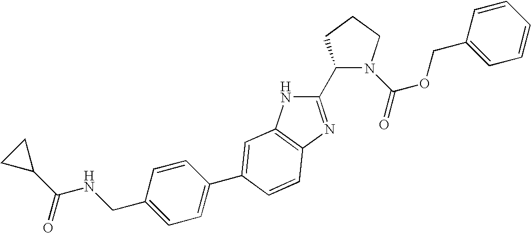 | 2-(6-{4-[(Cyclopropanecarbonyl-amino)-methyl]-phenyl}-1H-benzoimidazol-2-yl)-pyrrolidine-1-carboxylicacid benzyl ester |
| 4039 | 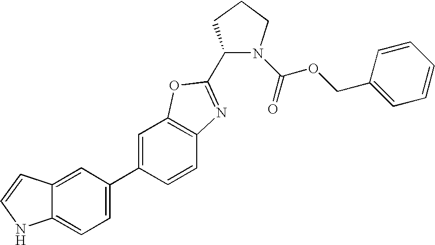 | 2-[6-(1H-Indol-5-yl)-benzooxazol-2-yl]-pyrrolidine-1-carboxylicacid benzyl ester |
| 4040 | 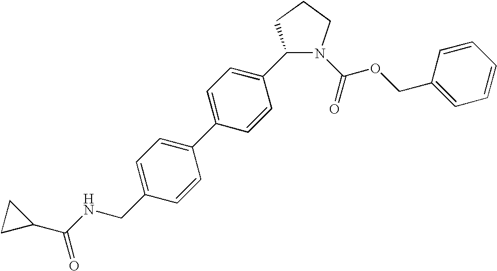 | 2-{4′-[(Cyclopropanecarbonyl-amino)-methyl]-biphenyl-4-yl}-pyrrolidine-1-carboxylic acid benzylester |
| 4041 | 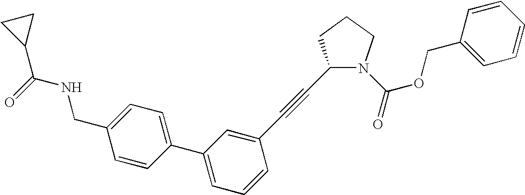 | 2-{4′-[(Cyclopropanecarbonyl-amino)-methyl]-biphenyl-3-ylethynyl}-pyrrolidine-1-carboxylic acid benzylester |
| 4042 | 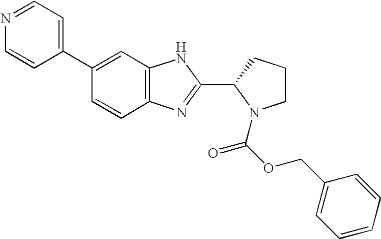 | 2-(6-Pyridin-4-yl-1H-benzoimidazol-2-yl)-pyrrolidine-1-carboxylicacid benzyl ester |
| 4043 | 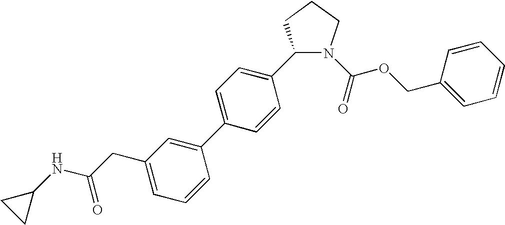 | 2-(3′-Cyclopropylcarbamoyl-methyl-biphenyl-4-yl)-pyrrolidine-1-carboxylicacid benzyl ester |
| 4044 | 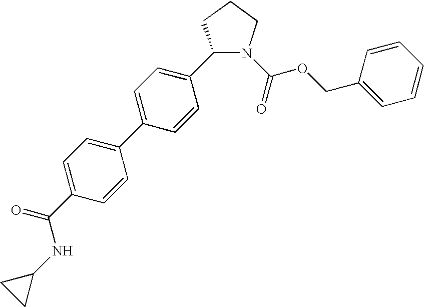 | 2-(4′-Cyclopropylcarbamoyl-biphenyl-4-yl)-pyrrolidine-1-carboxylicacid benzyl ester |
| 4045 | 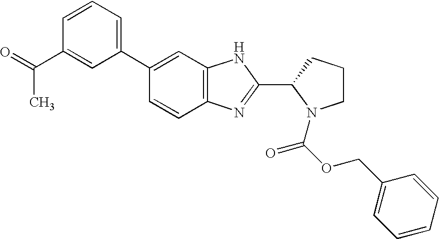 | 2-[6-(3-Acetyl-phenyl)-1H-benzoimidazol-2-yl]-pyrrolidine-1-carboxylicacid benzyl ester |
| 4046 | 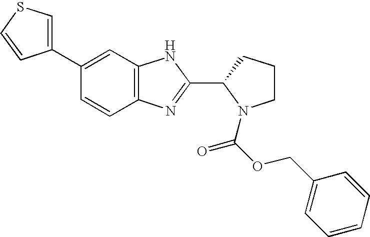 | 2-(6-Thiophen-3-yl-1H-benzoimidazol-2-yl)-pyrrolidine-1-carboxylicacid benzyl ester |
| 4047 | 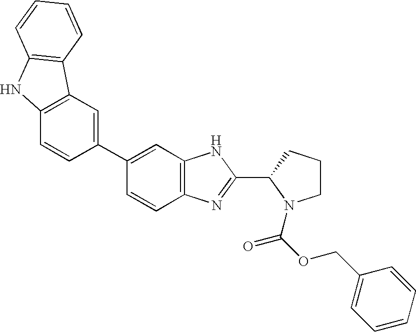 | 2-[6-(9H-Carbazol-3-yl)-1H-benzoimidazol-2-yl]-pyrrolidine-1-carboxylicacid benzyl ester |
| 4048 | 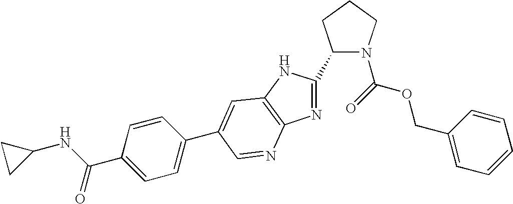 | 2-[6-(4-Cyclopropylcarbamoyl-phenyl)-1H-imidazo[4,5-b]pyridin-2-yl]-pyrrolidine-1-carboxylicacid benzyl ester |
| 4049 | 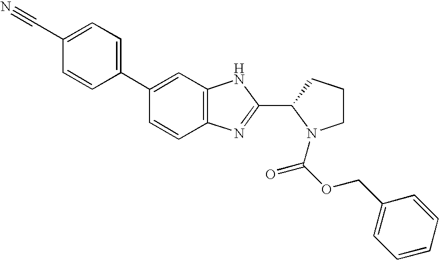 | 2-[6-(4-Cyano-phenyl)-1H-benzoimidazol-2-yl]-pyrrolidine-1-carboxylicacid benzyl ester |
| 4050 | 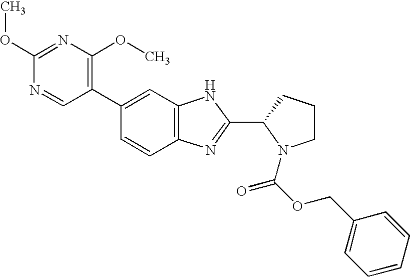 | 2-[6-(2,4-Dimethoxy-pyrimidin-5-yl)-1H-benzoimidazol-2-yl]-pyrrolidine-1-carboxylicacid benzyl ester |
| 4051 | 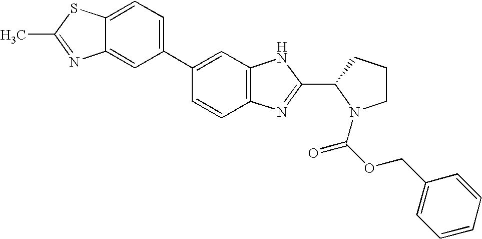 | 2-[6-(2-Methyl-benzothiazol-5-yl)-1H-benzoimidazol-2-yl]-pyrrolidine-1-carboxylicacid benzyl ester |
| 4052 | 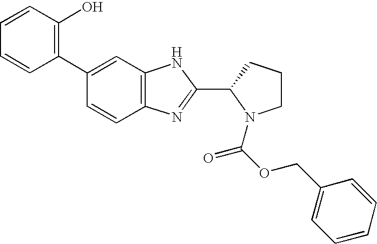 | 2-[6-(2-Hydroxy-phenyl)-1H-benzoimidazol-2-yl]-pyrrolidine-1-carboxylicacid benzyl ester |
| 4053 | 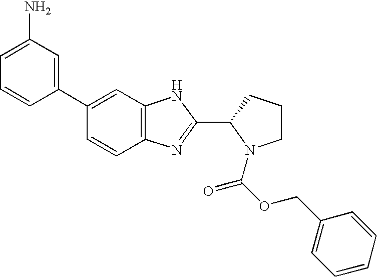 | 2-[6-(3-Amino-phenyl)-1H-benzoimidazol-2-yl]-pyrrolidine-1-carboxylicacid benzyl ester |
| 4054 | 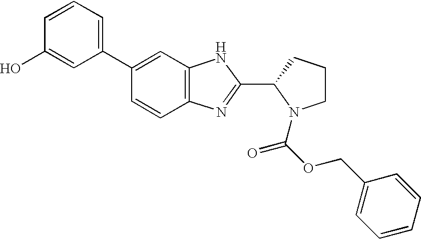 | 2-[6-(3-Hydroxy-phenyl)-1H-benzoimidazol-2-yl]-pyrrolidine-1-carboxylicacid benzyl ester |
| 4055 | 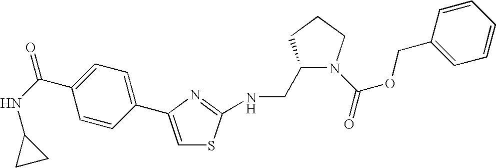 | 2-{[4-(4-Cyclopropylcarbamoyl-phenyl)-thiazol-2-ylamino]-methyl}-pyrrolidine-1-carboxylicacid benzyl ester |
| 4056 | 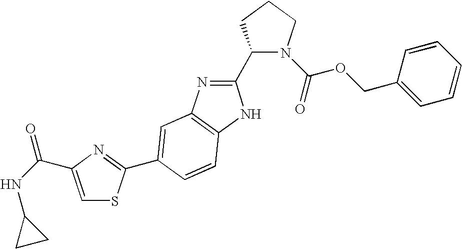 | 2-[5-(4-Cyclopropylcarbamoyl-thiazol-2-yl)-1H-benzoimidazol-2-yl]-pyrrolidine-1-carboxylicacid benzyl ester |
| 4057 | 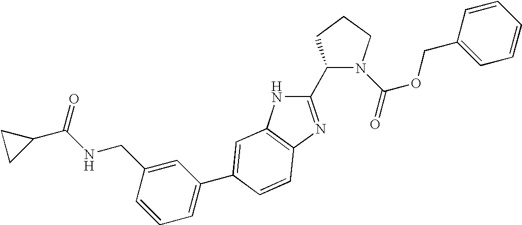 | 2-(6-{3-[(Cyclopropanecarbonyl-amino)-methyl]-phenyl}-1H-benzoimidazol-2-yl)-pyrrolidine-1-carboxylicacid benzyl ester |
| 4058 | 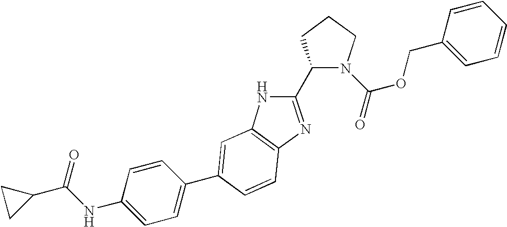 | 2-{6-[4-(Cyclopropanecarbonyl-amino)-phenyl]-1H-benzoimidazol-2-yl}-pyrrolidine-1-carboxylicacid benzylester |
| 4059 | 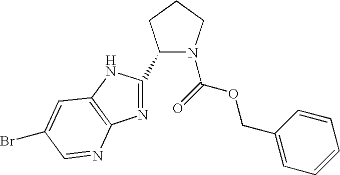 | 2-(6-Bromo-1H-imidazo[4,5-b]pyridin-2-yl)-pyrrolidine-1-carboxylic acid benzylester |
| 4060 | 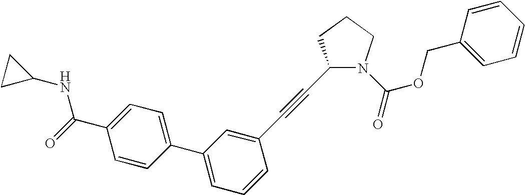 | 2-(4′-Cyclopropylcarbamoyl-biphenyl-3-ylethynyl)-pyrrolidine-1-carboxylicacid benzyl ester |

In one embodiment provided is a pharmaceutical composition comprising a pharmaceutically acceptable carrier and a therapeutically effective amount of a compound, stereoisomer, tautomer, pharmaceutically acceptable salt, or prodrug thereof of any one of Formula (I)-(III) or of the compounds in Table 1. In another embodiment provided is a method for treating a viral infection in a patient mediated at least in part by a virus in the Flaviviridae family of viruses, comprising administering to said patient such compositions. In some aspects, the viral infection is mediated by hepatitis C virus.

In other aspects, the administration of a therapeutically effective amount of the compounds and/or compositions of the invention are used in combination with one or more agents active against hepatitis C virus. These agent include an inhibitor of HCV proteases, HCV polymerase, HCV helicase, HCV NS4B protein, HCV entry, HCV assembly, HCV egress, HCV NS5A protein, or inosine 5′-monophosphate dehydrogenase. In other embodiments, the agent is interferon.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of compounds of Formula (I)-(III) may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula (I)-(III) in combination with at least one pharmaceutically acceptable excipient. Acceptable excipients are non-toxic, aid administration, and do not adversely affect the therapeutic benefit of the compound of Formula (I)-(III). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula (I)-(III) based on the total formulation, with the balance being one or more suitable pharmaceutical excipients. Preferably, the compound is present at a level of about 1-80 wt %. Representative pharmaceutical formulations containing a compound of Formula (I)-(III) are described below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV.

References herein to agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of Formula (I)-(III) and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of Formula (I)-(III) and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

The preparation of various intermediates of the invention may involved one or more amide bond forming reactions. A variety of amide coupling reagents may be used to from the amide bond, including the use of carbodiimides such as N—N′-dicyclohexylcarbodiimide (DCC), N—N′-diisopropylcarbodiimide (DIPCDI), and 1-ethyl-3-(3′-dimethylaminopropyl)carbodiimide (EDCI). The carbodiimides may be used in conjunction with additives such as benzotriazoles 7-aza-1-hydroxybenzotriazole (HOAt), 1-hydroxybenzotriazole (HOBt), and 6-chloro-1-hydroxybenzotriazole (Cl-HOBt).

Amide coupling reagents also include amininum and phosphonium based reagents. Aminium salts include N-[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino)methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP).

The amide formation step may be conducted in a polar solvent such as dimethylformamide (DMF) and may also include an organic base such as diisopropylethylamine (DIPEA).

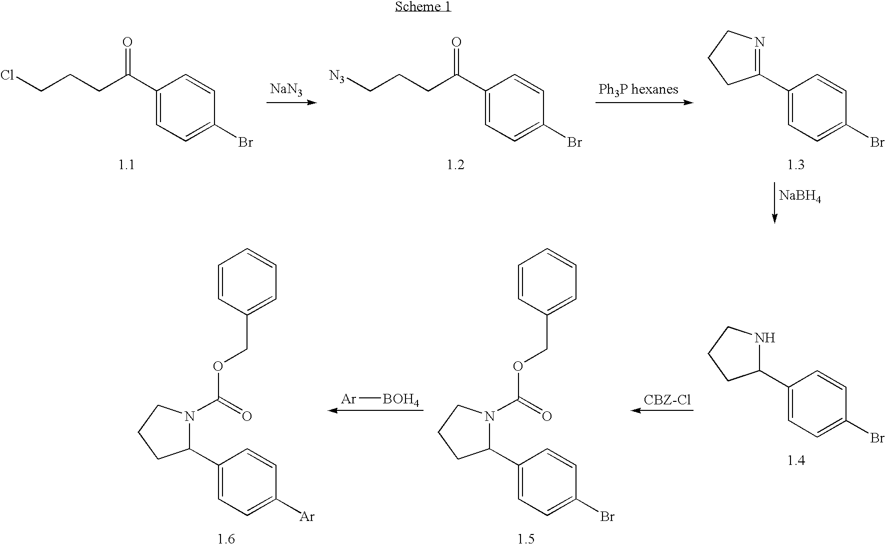

Scheme 1 shows a general synthesis of compounds of the invention where, for illustrative purposes ring A is a substituted phenyl ring, p is 0, and Z-R together form a benzyloxycarbonyl group. Reaction of halide 1.1 with an azide such as sodium azide gives 1.2 that is then treated with a reducing agent such as Ph₃P to form imine 1.3. Exposure of 1.3 to a reducing agent such as sodium borohydride gives amine 1.4 that is then functionalized such as by reacting carbonylbenzyloxy chloride to give carbamate 1.5. Reaction of intermediate 1.5 with an aryl boronic acid or a suitable coupling partner in a transition metal catalyzed cross-coupling reaction gives compound 1.6. Suitable transition metal catalysts include Pd based catalysts (e.g. Pd(PPh₃)₂Cl₂, Pd[P(Ph₃)]₄, etc.) such as those used in a Suzuki coupling. Intermediate 1.5 can also be further modified to yield the compounds of the invention using methods that will be apparent to one of skill in the art.

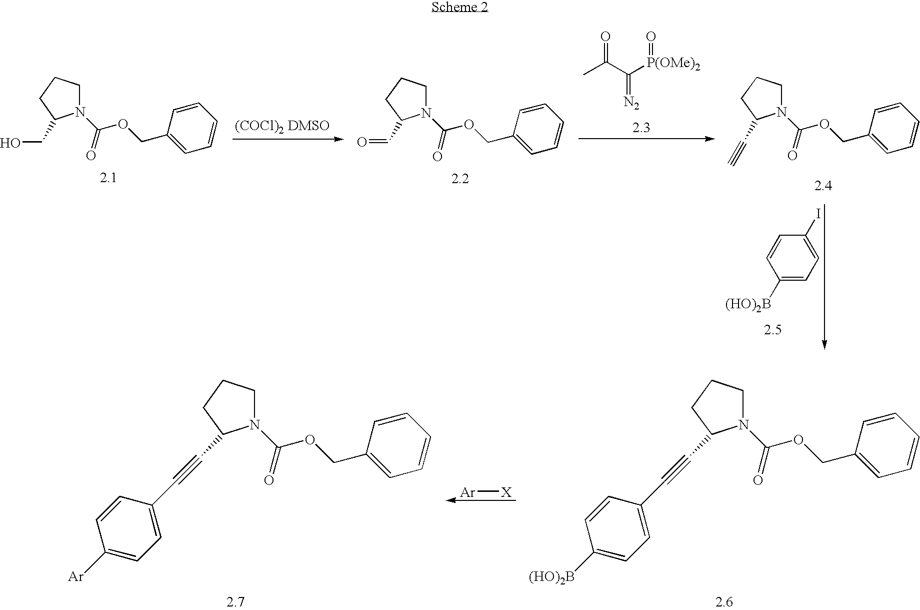

Scheme 2 shows the general synthesis of compounds where, for illustrative purposes, L¹ is C₂ alkynylene, ring A is a substituted phenyl ring, p is 0, and Z-R together form a benzyloxycarbonyl group. Prolinol 2.1 is exposed to oxidizing conditions such as a Swern oxidation to form aldehyde 2.2. Reaction of the aldehyde with Bestmann-Ohira reagent 2.3 in the presence of a base such as an alkoxide gives alkyne 2.4. Reaction of 2.4 with iodide 2.5 under Sonogashira coupling conditions (Cu(I), Et₃N, Pd(0)) gives boronic acid 2.4 that can then be coupled with Ar—X where X is a halide under Suzuki coupling conditions to give compound 2.7.

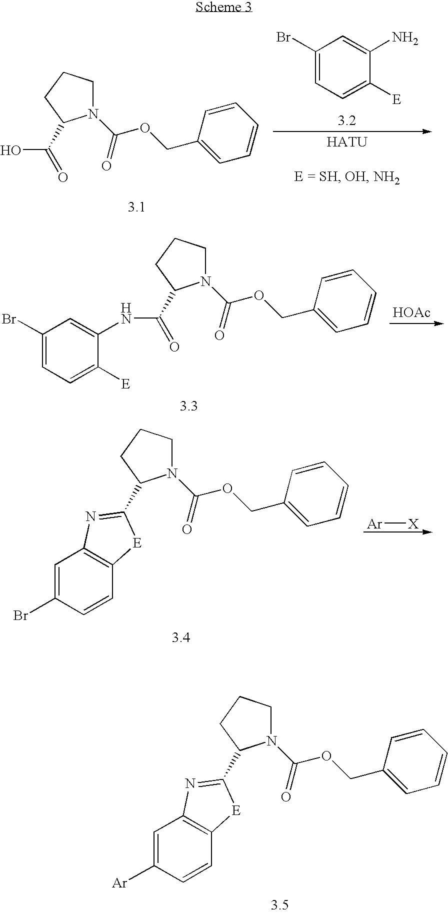

Scheme 3 shows the general synthesis of compounds where, for illustrative purposes, ring A is a substituted benzimidazole-2-yl, benzoxazole-2-yl, or benzthiazole-2-yl, p is 0, and Z-R together form a benzyloxycarbonyl group. Acid 3.1 is coupled with amine 3.2 in the presence of a coupling reagent such as HATU to give 3.2. Treatment of 3.3 with an acid such as acetic acid gives the cyclized halide 3.4 that can then be coupled with Ar—X where X is a halide under Suzuki coupling conditions to give compound 3.5.

The foregoing and other aspects of the present invention may be better understood in connection with the following representative examples.

### EXAMPLES

The examples below as well as throughout the application, the following abbreviations have the following meanings. If not defined, the terms have their generally accepted meanings.
atm=atmospheres
cm=centimeter
DMF=dimethylformamide
DIPEA=diisopropylethylamine
DMSO=dimethylsulfoxide
eq.=Equivalents
F.W.=Formula weight
g=gram
HATU=N-[(dimethylamino)-1H-1,2,3-triazolo[4,5-b]pyridine-1-ylmethylene]-N-methylmethanaminium hexafluorophosphate N-oxide
HPLC=high pressure liquid chromatography
KOAc=potassium acetate
L=liter
MeCN=acetonitrile
mg=milligram
mL=milliliter
mmol=millimole
MS=mass spectrum
TEA=triethylamine
TFA=trifluoroacetic acid
THF=tetrahydrofuran
TLC=thin layer chromatography
v/v=volume/volume
μL=microliter

### General Procedure A

A solution of 2-[6-(4,4,5,5-tetramethyl-[1,3,2]dioxaborolan-2-yl)-1H-benzoimidazol-2-yl]-pyrrolidine-1-carboxylic acid benzyl ester (100 mg, 0.220 mmol), aryl boronic acid (1 eq), Pd[P(Ph)₃]₄ (5 mol %, 13 mg), in methanol (2 mL), NaHCO₃ (sat. aq., 300 μL) and DMF (400 μL) was degassed and heated to 70° C. overnight in a sealed vial. The reaction was cooled, filtered and the solvents removed. The resulting mixture was redissolved in 5 mL of 90% DMF, 10% water with 0.1% TFA and purified by reverse phase HPLC to give the products.

### General Procedure B

A solution of 2-(6-bromo-1H-benzoimidazol-2-yl)-pyrrolidine-1-carboxylic acid benzyl ester (100 mg, 0.25 mmol), aryl boronic acid (1 eq), Pd[P(Ph)₃]₄ (5 mol %, 14 mg), in methanol (2 mL), NaHCO₃ (sat. aq., 300 μL) and DMF (400 μL) was degassed and heated to 70° C. overnight in a sealed vial. The reaction was cooled, filtered and the solvents removed. The resulting mixture was redissolved in 5 mL of 90% DMF, 10% water with 0.1% TFA and purified by reverse phase HPLC to give the products.

### General Procedure C

### (S)-2-(5-Bromo-2-hydroxy-phenylcarbamoyl)-pyrrolidine-1-carboxylic acid benzyl ester

Z-protected (S)-proline (664 mg) was dissolved in DMF (15 mL) and treated with HATU (1.1 eq. 1000 mg) and DIPEA (2.1 eq, 1 mL) and stirred for 15 minutes. Then 2-amino-4-bromo-phenol (1 eq, 500 mg) was added and the mixture stirred at ambient temperature overnight. The reaction was cooled, filtered and the solvents removed. The resulting mixture was redissolved in 5 mL of 90% DMF, 10% water with 0.1% TFA and purified by reverse phase HPLC to give the product. MS: 419.5 (M+H⁺)

### (S)-2-(5-Bromo-benzooxazol-2-yl)-pyrrolidine-1-carboxylic acid benzyl ester

2-(5-Bromo-2-hydroxy-phenylcarbamoyl)-pyrrolidine-1-carboxylic acid benzyl ester (from above) was dissolved in HOAc (50 mL) and heated to 110° C. for 12 hours. The reaction was cooled, filtered and the solvents removed. The resulting mixture was redissolved in 5 mL of 90% DMF, 10% water with 0.1% TFA and purified by reverse phase HPLC to give the product. MS: 401.5 (M+H⁺).

### (S)-2-(6-aryl-benzooxazol-2-yl)-pyrrolidine-1-carboxylic acid benzyl ester

A solution of (S)-2-(5-Bromo-benzooxazol-2-yl)-pyrrolidine-1-carboxylic acid benzyl ester (80 mg, 0.2 mmol), aryl boronic acid (1 eq), Pd[P(Ph)₃]₄ (5 mol %, 11 mg), in methanol (2 mL), NaHCO₃ (sat. aq., 300 μL) and DMF (400 μL) was degassed and heated to 70° C. overnight in a sealed vial. The reaction was cooled, filtered and the solvents removed. The resulting mixture was redissolved in 5 mL of 90% DMF, 10% water with 0.1% TFA and purified by reverse phase HPLC to give the products.

### General Procedure D

### 2-Amino-5-bromo-benzenethiol

6-Bromo-benzothiazol-2-ylamine (1 g) was dissolved in ethylene glycol (4 mL) treated with KOH aq. (4 mL, 10M) and heated to 125 deg. C. for 12 hours. The reaction was cooled, filtered, neutralized with HOAc and the solvents removed. The resulting mixture was redissolved in 5 mL of 90% DMF, 10% water with 0.1% TFA and purified by reverse phase HPLC to give the product. MS: 204.5 (M+H⁺).

### (S)-2-(2-Amino-5-bromo-phenylsulfanylcarbonyl)-pyrrolidine-1-carboxylic acid benzyl ester

Z-protected (S)-proline (1.35 g) was dissolved in DMF (5 mL) and treated with HBTU (1.1 eq. 2.3 g) and DIPEA (2.1 eq, 2.5 mL) and stirred for 15 minutes. Then 2-Amino-5-bromo-benzenethiol (1 eq, 1.1 g) was added and the mixture stirred at ambient temperature overnight. The reaction was cooled, filtered and the solvents removed. The resulting mixture was redissolved in 5 mL of 90% DMF, 10% water with 0.1% TFA and purified by reverse phase HPLC to give the product. MS: 435.5 (M+H⁺)

### (S)-2-(6-Bromo-benzothiazol-2-yl)-pyrrolidine-1-carboxylic acid benzyl ester

2-(2-Amino-5-bromo-phenylsulfanylcarbonyl)-pyrrolidine-1-carboxylic acid benzyl ester (from above) cyclized spontaneously in the refrigerator over one week. Used directly for the following reactions. MS: 417.5 (M+H⁺).

### (S)-2-(6-aryl-benzothiazol-2-yl)-pyrrolidine-1-carboxylic acid benzyl ester

A solution of (S)-2-(6-Bromo-benzothiazol-2-yl)-pyrrolidine-1-carboxylic acid benzyl ester (83 mg, 0.2 mmol), aryl boronic acid (1 eq), Pd[P(Ph)₃]₄ (5 mol %, 11 mg), in methanol (2 mL), NaHCO₃ (sat. aq., 300 μL) and DMF (400 μL) was degassed and heated to 70° C. overnight in a sealed vial. The reaction was cooled, filtered and the solvents removed. The resulting mixture was redissolved in 5 mL of 90% DMF, 10% water with 0.1% TFA and purified by reverse phase HPLC to give the products.

### Example 1

### (S)-2-[6-(3-Cyclopropylcarbamoyl-phenyl)-1H-benzoimidazol-2-yl]-pyrrolidine-1-carboxylic acid benzyl ester

### Compound 4001

### 3-Bromo-N-cyclopropyl-benzamide

3-Bromo-benzoic acid (2.5518 g, 12.7 mmol) and HATU (5.2230 g, 13.7 mmol) were dissolved in DMF (40 mL). DIPEA (4.644 mL, 26.7 mmol) was added, and the mixture was allowed to stir at room temperature for 15 minutes. Then cyclopropylamine (0.88 mL, 12.7 mmol) was added, and the reaction was stirred at ambient temperature overnight. The solvent was removed, and the crude was purified via silica gel chromatography to yield the desired product.

### (S)-2-[5-(4,4,5,5-Tetramethyl-[1,3,2]dioxaborolan-2-yl)-1H-benzoimidazol-2-yl]-pyrrolidine-1-carboxylic acid benzyl ester

To a solution of (S)-2-(5-Bromo-1H-benzoimidazol-2-yl)-pyrrolidine-1-carboxylic acid benzyl ester (3.5 g, 8.75 mmol) in DMSO (20 mL) was added KOAc (2.5 g), 4,4,5,5,4′,4′,5′,5′-Octamethyl-[2,2′]bi[[1,3,2]dioxaborolanyl] (5.8 g), NaHCO₃ (900 mg), PdP(Ph)₂Cl₂ (600 mg) and heated to 80 C overnight. The mixture was precipitated by pouring into 20 mL of water, filtered, loaded onto a silica column and the product eluted at 20% EtOAc in hexanes. MS: 448 (M+H⁺).

### (S)-2-[5-(3-Cyclopropylcarbamoyl-phenyl)-1H-benzoimidazol-2-yl]-pyrrolidine-1-carboxylic acid benzyl ester

### Compound 4001

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (90 mg, 0.20 mmol), 3-bromo-N-cyclopropyl-benzamide (48.6 mg, 0.20 mmol), and Pd[P(Ph)₃]₄ (14.5 mg, 6.2 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 15.0 mg. MS: 481.2 (M+H⁺); H¹ NMR (DMSO-d₆): δ(ppm) 0.52-0.74 (m, 4H), 1.88-2.18 (m, 3H), 2.78-2.89 (m, 1H), 3.42-3.74 (m, 2H), 4.78-5.13 (m, 2H), 5.16-5.29 (m, 1H), 6.78-7.09 (m, 2H), 7.24-7.40 (m, 2H), 7.48+7.58 (m, 1H), 7.67-7.96 (m, 6H), 8.04-8.10 (s, 1H), 8.49-8.58 (m, 1H).

### Example 2

### 2-(4′-Cyclopropylcarbamoylmethyl-biphenyl-4-yl)-pyrrolidine-1-carboxylic acid benzyl ester

### Compound 4002

### 2-(4-Bromo-phenyl)-N-cyclopropyl-acetamide

(4-Bromo-phenyl)-acetic acid (632.3 mg, 2.9 mmol) and DCC (609.2 mg, 3.0 mmol) were dissolved in dichloromethane (15 mL) and stirred at ambient temperature for 5 minutes. Then cyclopropylamine (204.5 μL, 3.0 mmol) was added, and the reaction was stirred at ambient temperature overnight. The reaction was filtered, and the solvent was removed. The resulting mixture was redissolved in DMF (10 mL) and purified by reverse phase HPLC to give the desired product.

### 4-Azido-1-(4-bromo-phenyl)-butan-1-one

A solution of 1-(4-bromo-phenyl)-4-chloro-butan-1-one (2.61 g, 10 mmol) in DMSO (10 mL) was treated with NaI (100 mg) and NaN₃ (3 eq., 2 g). The mixture was heated to 60° C. overnight and then cooled. Water (30 mL) was added and the solution extracted with EtOAc (2×, 20 mL). The organics were washed with brine (1×), dried (Na₂SO₄) and the solvents removed. MS: 268 (M+H⁺).

### 5-(4-Bromo-phenyl)-3,4-dihydro-2H-pyrrole

A suspension of 4-azido-1-(4-bromo-phenyl)-butan-1-one (2.7 g, 10 mmol) in hexanes (50 mL) was treated with triphenyl phosphine (1 eq., 2.62 g) and stirred at room temperature overnight. The suspension was filtered, and washed with cold Et₂O. The ether phase was diluted with 1 volume of hexanes and filtered. The organic phase was stripped of solvents and the crude product used directly in the next reaction. MS: 224 (M+H⁺).

### 2-(4-Bromo-phenyl)-pyrrolidine

5-(4-Bromo-phenyl)-3,4-dihydro-2H-pyrrole (10 mmol) was dissolved in MeOH (w/20% HOAc, 5 mL) and cooled to −40° C. NaBH₄ (2 eq, 760 mg) was added and the mixture warmed to room temperature. After 2 hrs the reaction was acidified with HCl (2M, 50 mL) extracted with ether (2×50 mL), the aqueous layer was basified with NaOH (2M in water, 100 mL) and extracted with EtOAc (2×50 mL). The organic phase was dried with Na₂SO₄ and concentrated to an oil. MS: 226 (M+H⁺).

### 2-(4-Bromo-phenyl)-pyrrolidine-1-carboxylic acid benzyl ester

A solution of 5-(4-bromo-phenyl)-3,4-dihydro-2H-pyrrole (661 mg, 3 mmol) in DMF (15 mL) at 0° C. was treated with DIPEA (1.5 eq., 781 μL) then carbonylbenzyloxychloride (1.3 eq., 540 μL). After 2 hours the solvents were removed the crude mixture was dissolved in DMF, acidified with 0.1% TFA and purified on reversed phase HPLC to give the product. MS: 360 (M+H⁺).

### N-Cyclopropyl-2-[4-(4,4,5,5-tetramethyl-[1,3,2]dioxaborolan-2-yl)-phenyl]-acetamide

A solution of 4,4,5,5,4′,4′,5′,5′-octamethyl-[2,2′]bi[[1,3,2]dioxaborolanyl] (3.7877 g, 14.92 mmol), 2-(4-bromo-phenyl)-N-cyclopropyl-acetamide (1.2612 g, 4.96 mmol), potassium acetate (1.4608 g, 14.88 mmol), and Pd(PPh₃)₂Cl₂ (0.3490 g, 10.0 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-(4′-Cyclopropylcarbamoylmethyl-biphenyl-4-yl)-pyrrolidine-1-carboxylic acid benzyl ester

### Compound 4002

A solution of N-cyclopropyl-2-[4-(4,4,5,5-tetramethyl-[1,3,2]dioxaborolan-2-yl)-phenyl]-acetamide (66.4 mg, 0.22 mmol), 2-(4-bromo-phenyl)-pyrrolidine-1-carboxylic acid benzyl ester (84.0 mg, 0.23 mmol), and Pd[P(Ph)₃]₄ (12.4 mg, 4.6 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 44.9 mg. MS: 455.2 (M+H⁺); H¹ NMR (DMSO-d₆): δ(ppm) 0.37-0.46 (m, 2H), 0.58-0.68 (m, 2H), 1.68-1.93 (m, 3H), 2.20-2.46 (m, 1H), 2.56-2.68 (m, 1H), 3.34-3.42 (s, 2H), 3.48-3.73 (m 2H), 4.81-5.12 (m, 3H), 6.80-6.90 (m, 1H), 7.05-7.40 (m, 8H), 7.49-7.63 (m, 4H), 8.13-8.20 (d, 1H).

### Example 3

### (S)-2-[6-(3-Cyclopropylcarbamoylmethyl-phenyl)-1H-benzoimidazol-2-yl]-pyrrolidine-1-carboxylic acid benzyl ester

### Compound 4003

### 2-(3-Bromo-phenyl)-N-cyclopropyl-acetamide

(3-Bromo-phenyl)-acetic acid (636.0 mg, 3.0 mmol) and DCC (608.2 mg, 2.9 mmol) were dissolved in dichloromethane (15 mL) and stirred at ambient temperature for 5 minutes. Then cyclopropylamine (204.5 μL, 3.0 mmol) was added, and the reaction was stirred at ambient temperature overnight. The reaction was filtered, and the solvent was removed. The resulting mixture was redissolved in DMF (10 mL) and purified by reverse phase HPLC to give the desired product.

### (S)-2-[6-(3-Cyclopropylcarbamoylmethyl-phenyl)-1H-benzoimidazol-2-yl]-pyrrolidine-1-carboxylic acid benzyl ester

### Compound 4003

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (90 mg, 0.20 mmol), 2-(3-bromo-phenyl)-N-cyclopropyl-acetamide (51.8 mg, 0.20 mmol), and Pd[P(Ph)₃]₄ (13.5 mg, 5.8 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 15.0 mg. MS: 495.2 (M+H⁺); H¹ NMR (DMSO-d₆): δ(ppm) 0.30-0.55 (m, 2H), 0.53-0.78 (m, 2H), 1.92-2.21 (m, 3H), 2.55-2.68 (m, 1H), 3.55-3.80 (m, 2H), 4.81-5.19 (m, 2H), 5.19-5.32 (m, 1H), 7.02-7.13 (m, 2H), 7.21-7.89 (m, 10H), 8.16-8.23 (m, 1H).

### Example 4

### (S)-2-[4′-(Cyclopropanecarbonyl-amino)-biphenyl-3-ylethynyl]-pyrrolidine-1-carboxylic acid benzyl ester

### Compound 4004

A solution of(S)-2-(3-boronic acid-phenylethynyl)-pyrrolidine-1-carboxylic acid benzyl ester (62.5 mg, 0.18 mmol), cyclopropanecarboxylic acid (4-bromo-phenyl)-amide (Example 58, 43.5 mg, 0.18 mmol), and Pd[P(Ph)₃]₄ (12.7 mg, 6.1 mol %) in methanol (2 mL), NaHCO₃ (sat. aq., 300 μL), and DMF (1.6 mL) was degassed and heated to 70° C. overnight in a sealed vial. The reaction was cooled, filtered, and purified by reverse phase HPLC to give the desired product. Yield 21.8 mg. MS: 465.2 (M+H⁺); H¹ NMR (DMSO-d₆): δ(ppm) 0.73-0.85 (m, 4H), 1.70-2.30 (m, 5H), 4.71-4.83 (m, 1H), 4.80-5.31 (m, 2H), 7.17-7.71 (m, 13H), 10.24-10.31 (2, 1H).

### Example 5

### (S)-2-[6-(1H-Indol-5-yl)-benzothiazol-2-yl]-pyrrolidine-1-carboxylic acid benzyl ester

### Compound 4005

From 1 eq. 1H-indole 5-boronic acid following General Procedure D. Yield 44 mg. MS: 454.5 (M+H⁺); H¹-NMR (DMSO-d₆): δ(ppm) 11.1 (s, 1H), 8.3 (s, 1H), 7.9-7.7 (m, 3H), 7.5-7.3 (m, 6H), 7.1-6.9 (m, 2H), 6.5 (s, 1H), 5.3 (m, 1H), 5.2-4.8 (m, 3H), 3.6-3.5 (m, 4H), 2.39 (m, 1H), 2.12-1.98 (m, 3H).

### Example 6

### (S)-4-[5-(4-Cyclopropylcarbamoyl-phenyl)-1H-benzoimidazol-2-yl]-2-pyridin-4-yl-thiazolidine-3-carboxylic acid benzyl ester

### Compound 4006

### (S)-4-(2-Amino-4-bromo-phenylcarbamoyl)-2-pyridin-4-yl-thiazolidine-3-carboxylic acid benzyl ester

2-Pyridin-4-yl-thiazolidine-3,4-dicarboxylic acid 3-benzyl ester (172 mg) was dissolved in DMF (15 mL) and treated with PyBrOP (1.1 eq. 256 mg) and DIPEA (2.1 eq, 200 μL) and stirred for 15 minutes. Then 4-bromo-benzene-1,2-diamine (1 eq, 102 mg) was added and the mixture stirred at ambient temperature overnight. The reaction was cooled, filtered and the solvents removed. The resulting mixture was redissolved in 5 mL of 90% DMF, 10% water with 0.1% TFA and purified by reverse phase HPLC to give the product. MS: 513.5 (M+H⁺)

### (S)-4-(5-Bromo-1H-benzoimidazol-2-yl)-2-pyridin-4-yl-thiazolidine-3-carboxylic acid benzyl ester

(S)-4-(2-Amino-4-bromo-phenylcarbamoyl)-2-pyridin-4-yl-thiazolidine-3-carboxylic acid benzyl ester (from above) was dissolved in HOAc (10 mL) and heated to 90° C. for 3 hours. The reaction was cooled, filtered and the solvents removed. The resulting mixture was redissolved in 5 mL of 90% DMF, 10% water with 0.1% TFA and purified by reverse phase HPLC to give the product. MS: 495.5 (M+H⁺).

### (S)-4-[5-(4-Cyclopropylcarbamoyl-phenyl)-1H-benzoimidazol-2-yl]-2-pyridin-4-yl-thiazolidine-3-carboxylic acid benzyl ester

### Compound 4006

A solution of (S)-4-(5-bromo-1H-benzoimidazol-2-yl)-2-pyridin-4-yl-thiazolidine-3-carboxylic acid benzyl ester (49.5 mg, 0.1 mmol), N-cyclopropyl-4-(4,4,5,5-tetramethyl-[1,3,2]dioxaborolan-2-yl)-benzamide (Example 10, 29 mg, 1 eq), Pd[P(Ph)₃]₄ (5 mol %, 11 mg), in methanol (2 mL), NaHCO₃ (sat. aq., 300 μL) and DMF (400 μL) was degassed and heated to 70° C. overnight in a sealed vial. The reaction was cooled, filtered and the solvents removed. The resulting mixture was redissolved in 5 mL of 90% DMF, 10% water with 0.1% TFA and purified by reverse phase HPLC to give the product. Yield 33.5 mg. MS: 576.5 (M+H⁺); H¹-NMR (DMSO-d₆): δ (ppm) 8.8-8.4 (m, 3H), 8.0-7.6 (m, 6H), 7.3-6.9 (m, 5H), 6.4-6.0 (m, 2H), 5.2-4.8 (m, 3H), 3.7 (m, 2H), 3.4 (m, 2H), 2.87 (m, 1H), 0.7-0.6 (m, 4H).

### Example 7

### (S)-2-(6-Phenyl-1H-benzoimidazol-2-yl)-pyrrolidine-1-carboxylic acid benzyl ester

### Compound 4007

From 1 eq of bromo-benzene following General Procedure A. Yield 14.0 mg. MS: 398.5 (M+H⁺); H¹-NMR (DMSO-d₆): δ(ppm) 7.9-7.7 (m, 5H), 7.48 (s, 2H), 7.3 (m, 3H), 7.0-6.8 (m, 3H), 5.2-4.8 (m, 3H), 3.70 (m, 1H), 3.55 (m, 1H), 2.02 (m, 4H).

### Example 8

### (S)-2-[6-(4-Amino-phenyl)-1H-benzoimidazol-2-yl]-pyrrolidine-1-carboxylic acid benzyl ester

### Compound 4008

From 1 eq. of 4-bromo-phenylamine following General Procedure A. Yield 6.6 mg. MS: 412.5 (M+H⁺); H¹-NMR (DMSO-d₆): δ(ppm) 7.85-7.6 (m, 4H), 7.35 (m, 3H), 7.1 (m, 2H), 7.0-6.8 (m, 3H), 5.2-4.8 (m, 3H), 3.70 (m, 1H), 3.55 (m, 1H), 2.02 (m, 4H).

### Example 9

### 2-[3′-(Cyclopropanecarbonyl-amino)-biphenyl-4-yl]-pyrrolidine-1-carboxylic acid benzyl ester

### Compound 4009

### Cyclopropanecarboxylic acid [3-(4,4,5,5-tetramethyl-[1,3,2]dioxaborolan-2-yl)-phenyl]-amide

A solution of 4,4,5,5,4′,4′,5′,5′-octamethyl-[2,2′]bi[[1,3,2]dioxaborolanyl] (3.9783 g, 15.67 mmol), cyclopropanecarboxylic acid (3-bromo-phenyl)-amide (Example 37, 1.2515 g, 5.21 mmol), potassium acetate (1.5250 g, 15.54 mmol), and Pd(PPh₃)₂Cl₂ (0.3646 g, 10.0 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-[3′-(Cyclopropanecarbonyl-amino)-biphenyl-4-yl]-pyrrolidine-1-carboxylic acid benzyl ester

### Compound 4009

A solution of cyclopropanecarboxylic acid [3-(4,4,5,5-tetramethyl-[1,3,2]dioxaborolan-2-yl)-phenyl]-amide (65.5 mg, 0.23 mmol), 2-(4-bromo-phenyl)-pyrrolidine-1-carboxylic acid benzyl ester (83.3 mg, 0.23 mmol), and Pd[P(Ph)₃]₄ (12.4 mg, 4.6 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 14.2 mg. MS: 441.2 (M+H⁺); H¹ NMR (DMSO-d₆): δ(ppm) 0.75-0.90 (m, 4H), 1.68-1.94 (m, 4H), 2.21-2.45 (m, 1H), 3.47-3.74 (m, 2H), 4.80-5.13 (m, 3H), 6.78-6.89 (m, 1H), 7.05-7.59 (m, 11H), 7.84-7.97 (d, 1H), 10.22-10.33 (s, 1H).

### Example 10

### (S)-2-[6-(4-Cyclopropylcarbamoyl-phenyl)-1H-benzoimidazol-2-yl]-2,3-dihydro-indole-1-carboxylic acid benzyl ester

### Compound 4010

### 4-Bromo-N-cyclopropyl-benzamide

4-Bromo-benzoic acid (2.5116 g, 12.5 mmol) and HATU (5.2213 g, 13.7 mmol) were dissolved in DMF (40 mL). DIPEA (4.572 mL, 26.2 mmol) was added, and the mixture was allowed to stir at ambient temperature for 15 minutes. Then cyclopropylamine (0.866 mL, 12.5 mmol) was added, and the reaction was stirred at ambient temperature overnight. The solvent was removed, and the crude was purified via silica gel chromatography to give the desired product.

### N-Cyclopropyl-4-(4,4,5,5-tetramethyl-[1,3,2]dioxaborolan-2-yl)-benzamide

A solution of 4,4,5,5,4′,4′,5′,5′-octamethyl-[2,2′]bi[[1,3,2]dioxaborolanyl] (3.9837 g, 15.67 mmol), 4-bromo-N-cyclopropyl-benzamide (1.2516 g, 5.21 mmol), potassium acetate (1.5353 g, 15.64 mmol), and Pd(PPh₃)₂Cl₂ (0.3677 g, 10.0 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### (S)-2-(2-Amino-4-bromo-phenylcarbamoyl)-2,3-dihydro-indole-1-carboxylic acid benzyl ester & (S)-2-(2-Amino-5-bromo-phenylcarbamoyl)-2,3-dihydro-indole-1-carboxylic acid benzyl ester

(S)-2,3-Dihydro-indole-1,2-dicarboxylic acid 1-benzyl ester (394.7 mg, 1.33 mmol) was dissolved in DMF (10 mL). DIPEA (465 μL, 2.67 mmol) was added followed by HATU (504.8 mg, 1.33 mmol). The reaction was stirred at ambient temperature for 15 minutes. Then 4-bromo-benzene-1,2-diamine (250.1 mg, 1.34 mmol) was added, and the reaction was stirred at ambient temperature for 1 hour. The reaction was filtered and purified by reverse phase HPLC.

### (S)-2-(6-Bromo-1H-benzoimidazol-2-yl)-2,3-dihydro-indole-1-carboxylic acid benzyl ester

A solution of (S)-2-(2-amino-4-bromo-phenylcarbamoyl)-2,3-dihydro-indole-1-carboxylic acid benzyl ester and (S)-2-(2-Amino-5-bromo-phenylcarbamoyl)-2,3-dihydro-indole-1-carboxylic acid benzyl ester (619 mg, 1.33 mmol) in glacial acetic acid (5 mL) was heated to 110° C. in a sealed vial for 2.5 hours. The acetic acid was removed, and the resulting mixture was filtered and purified by reverse phase HPLC to give the desired product.

### (S)-2-[6-(4-Cyclopropylcarbamoyl-phenyl)-1H-benzoimidazol-2-yl]-2,3-dihydro-indole-1-carboxylic acid benzyl ester

### Compound 4010

A solution of N-cyclopropyl-4-(4,4,5,5-tetramethyl-[1,3,2]dioxaborolan-2-yl)-benzamide (75.9 mg, 0.26 mmol), (S)-2-(6-bromo-1H-benzoimidazol-2-yl)-2,3-dihydro-indole-1-carboxylic acid benzyl ester (60 mg, 0.13 mmol), and Pd[P(Ph)₃]₄ (8.3 mg, 5.4 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. The product was then converted to the HCl salt. After dissolving the product in a minimum amount of acetonitrile and cooling the solution in dry ice, 2.0M HCl in diethyl ether was added until precipitate crashed out of solution. The mixture was centrifuged, and the liquid was decanted. Additional cold diethyl ether was added, and the mixture was again centrifuged and the liquid decanted. The resulting solid was dried to give the HCl salt of the desired product. Yield 7.9 mg. MS: 529.2 (M+H⁺); H¹ NMR (DMSO-d₆): δ(ppm) 0.55-0.77 (m, 4H), 2.80-2.93 (m, 1H), 3.32-3.45 (m, 1H), 3.75-3.90 (m, 1H), 5.96-6.08 (m, 1H), 6.87-7.33 (m, 6H), 7.68-8.00 (m, 8H), 8.48-8.54 (d, 1H).

### Example 11

### (S)-2-(6-Naphthalen-2-yl-1H-benzoimidazol-2-yl)-pyrrolidine-1-carboxylic acid benzyl ester

### Compound 4011

From 1 eq. of 2-Bromo-naphthalene following General Procedure A. Yield 5.4 mg. MS: 448.5 (M-NHCOCH₃); H¹-NMR (DMSO-d₆): δ(ppm) 8.2 (s, 1H), 8.0-7.6 (m, 7H), 7.5 (m, 2H), 7.3 (m, 2H), 7.0-6.8 (m, 3H), 5.2-4.8 (m, 3H), 3.70 (m, 1H), 3.55 (m, 1H), 2.02 (m, 4H).

### Example 12

### (S)-2-[6-(5-Nitro-furan-2-yl)-1H-benzoimidazol-2-yl]-pyrrolidine-1-carboxylic acid benzyl ester

### Compound 4012

From 1 eq. of 2-Bromo-5-nitro-furan following General Procedure A. Yield 8 mg. MS: 433.4 (M+H⁺); H¹-NMR (DMSO-d₆): δ(ppm) 8.1-7.7 (m, 3H), 7.5 (m, 1H), 7.3 (m, 3H), 7.0-6.8 (m, 3H), 5.2-4.8 (m, 3H), 3.70 (m, 1H), 3.55 (m, 1H), 2.02 (m, 4H).

### Example 13

### (S)-2-(6-Quinolin-3-yl-1H-benzoimidazol-2-yl)-pyrrolidine-1-carboxylic acid benzyl ester

### Compound 4013

From 1 eq. of 3-Bromo-quinoline following General Procedure A. Yield 24 mg. MS: 449.5 (M+H⁺); H¹-NMR (DMSO-d₆): δ(ppm) 9.5 (m, 1H), 9.1 (m, 1H), 8.3-7.7 (m, 6H), 7.5 (m, 1H), 7.3 (m, 3H), 7.0-6.8 (m, 2H), 5.2-4.8 (m, 3H), 3.70 (m, 1H), 3.55 (m, 1H), 2.02 (m, 4H).

### Example 14

### (S)-2-[6-(1H-Indol-7-yl)-1H-benzoimidazol-2-yl]-pyrrolidine-1-carboxylic acid benzyl ester

### Compound 4014

From 1 eq. of 7-Bromo-1H-indole following General Procedure A. Yield 16 mg. MS: 437.5 (M+H⁺); H¹-NMR (DMSO-d₆): δ(ppm) 7.9-7.5 (s, 5H), 7.3 (m, 4H), 7.1-6.8 (m, 4H), 6.5 (m, 1H), 5.2-4.8 (m, 3H), 3.70 (m, 1H), 3.55 (m, 1H), 2.02 (m, 4H).

### Example 15

### (S)-2-[6-(4-Trifluoromethyl-phenyl)-1H-benzoimidazol-2-yl]-pyrrolidine-1-carboxylic acid benzyl ester

### Compound 4015

From 1 eq. of 4-trifluoromethyl phenylboronic acid following General Procedure B. Yield 25 mg. MS: 466.5 (M+H⁺); H¹-NMR (DMSO-d₆): δ(ppm) 8.2-8.4 (m, 4H), 7.9 (m, 1H), 7.7 (m, 1H), 7.75 (s, 1H), 7.3 (m, 3H), 7.0-6.8 (m, 3H), 5.2-4.8 (m, 3H), 3.81 (s, 3H), 3.70 (m, 1H), 3.55 (m, 1H), 2.02 (m, 4H).

### Example 16

### (S)-2-[6-(3-Methoxy-phenyl)-1H-benzoimidazol-2-yl]-pyrrolidine-1-carboxylic acid benzyl ester

### Compound 4016

From 1 eq. of 3-Methoxy-phenylboronic acid following General Procedure B. Yield 24 mg. MS: 428.5 ((M+H⁺); H¹-NMR (DMSO-d₆): δ(ppm) 7.9-7.7 (m, 5H), 7.4-7.2 (m, 5H), 7.0-6.8 (m, 2H), 5.2-4.8 (m, 3H), 3.84 (s, 3H), 3.70 (m, 1H), 3.55 (m, 1H), 2.02 (m, 4H).

### Example 17

### (S)-2-[6-(2,4-Dimethoxy-phenyl)-1H-benzoimidazol-2-yl]-pyrrolidine-1-carboxylic acid benzyl ester

### Compound 4017

From 1 eq. of 2,4-Dimethoxy-phenyl-lboronic acid following General Procedure B. Yield 15 mg. MS: 458.5 (M+H⁺); H¹-NMR (DMSO-d₆): δ(ppm) 7.7 (m, 3H), 7.5 (m, 1H), 7.4-7.25 (m, 4H), 7.0-6.8 (m, 3H), 6.65 (m, 2H), 5.2-4.8 (m, 3H), 3.81 (s, 3H), 3.77 (s, 3H), 3.70 (m, 1H), 3.55 (m, 1H), 2.02 (m, 4H).

### Example 18

### (S)-2-[6-(1H-Indol-6-yl)-1H-benzoimidazol-2-yl]-pyrrolidine-1-carboxylic acid benzyl ester

### Compound 4018

From 1 eq. of 1H-indole 6-lboronic acid following General Procedure B. Yield 36 mg. MS: 437.5 (M-NHCOCH₃); H¹-NMR (DMSO-d₆): δ(ppm) 7.9-7.6 (m, 5H), 7.3 (m, 4H), 7.0-6.8 (m, 2H), 6.5 (m, 1H), 5.2-4.8 (m, 3H), 3.81 (s, 3H), 3.70 (m, 1H), 3.55 (m, 1H), 2.02 (m, 4H).

### Example 19

### (S)-2-[6-(4-Cyclopropylcarbamoyl-phenyl)-benzooxazol-2-yl]-pyrrolidine-1-carboxylic acid benzyl ester

### Compound 4019

From 1 eq. of N-cyclopropyl-4-(4,4,5,5-tetramethyl-[1,3,2]dioxaborolan-2-yl)-benzamide (Example 10) following General Procedure C. Yield 22 mg. MS: 482.5 (M-NHCOCH₃); H¹-NMR (DMSO-d₆): δ(ppm) 8.4 (m, 1H), 8.0 (m, 1H), 7.9-7.6 (m, 5H), 7.3 (m, 2H), 7.1-6.8 (m, 2H), 5.2-4.8 (m, 3H), 2.8 (m, 1H), 2.42 (m, 1H), 2.02 (m, 3H), 0.7-0.6 (m, 4H)

### Example 20

### (S)-2-[6-(4-Cyclopropylcarbamoyl-phenyl)-1H-benzoimidazol-2-yl]-pyrrolidine-1-carboxylic acid benzyl ester

### Compound 4020

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (93.5 mg, 0.33 mmol), 4-bromo-N-cyclopropyl-benzamide (Example 10, 137.4 mg, 0.34 mmol), and Pd[P(Ph)₃]₄ (19.2 mg, 5.1 mol %) in methanol (4 mL), NaHCO₃ (sat. aq., 600 μL), and DMF (800 μL) was degassed and heated to 70° C. overnight in a sealed vial. The reaction was cooled, filtered, and purified by reverse phase HPLC to give the desired product. The product was then converted to the HCl salt. After dissolving the product in a minimum amount of acetonitrile and cooling the solution in dry ice, 2.0M HCl in diethyl ether was added until precipitate crashed out of solution. The mixture was centrifuged, and the liquid was decanted. Additional cold diethyl ether was added, and the mixture was again centrifuged and the liquid decanted. The resulting solid was dried to give the HCl salt of the desired product. Yield 32.2 mg. MS: 481.2 (M+H⁺); H¹ NMR (DMSO-d₆): δ(ppm) 0.54-0.76 (m, 4H), 1.92-2.27 (m, 3H), 2.81-2.93 (m, 1H), 3.38-3.85 (m, 2H), 4.80-5.17 (m, 2H), 5.25-5.40 (m, 1H), 6.83-7.10 (m, 2H), 7.26-7.43 (m, 2H), 7.74-8.03 (m, 8H), 8.49-8.57 (m, 1H).

### Example 21

### (S)-2-[6-(4-Cyclopropylcarbamoylmethyl-phenyl)-1H-benzoimidazol-2-yl]-pyrrolidine-1-carboxylic acid benzyl ester

### Compound 4021

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (90 mg, 0.20 mmol), 2-(4-bromo-phenyl)-N-cyclopropyl-acetamide (Example 2, 51.0 mg, 0.20 mmol), and Pd[P(Ph)₃]₄ (14.3 mg, 6.2 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 15.0 mg. MS: 495.2 (M+H⁺); H¹ NMR (DMSO-d₆): δ(ppm) 0.35-0.45 (m, 2H), 0.53-0.67 (m, 2H), 1.90-2.20 (m, 3H), 2.55-2.69 (m, 1H), 3.45-3.81 (m, 2H), 4.79-5.18 (m, 2H), 5.18-5.32 (m, 1H), 6.80-7.12 (m, 2H), 7.24-7.41 (m, 4H), 7.50-7.97 (m, 6H), 8.11-8.22 (m, 1H).

### Example 22

### (S)-2-[6-(4-Cyclopropylcarbamoyl-phenyl)-benzothazol-2-yl]-pyrrolidine-1-carboxylic acid benzyl ester

### Compound 4022

From 1 eq. of N-cyclopropyl-4-(4,4,5,5-tetramethyl-[1,3,2]dioxaborolan-2-yl)-benzamide (Example 10) following General Procedure D. Yield 6.5 mg. MS: 498 (M-NHCOCH₃); H¹-NMR (DMSO-d₆): δ(ppm) 8.5 (m, 1H), 8.4 (m, 1H), 8.0-7.8 (m, 6H), 7.3 (m, 2H), 7.1-6.8 (m, 2H), 5.3 (m, 1H), 5.2-4.8 (m, 2H), 3.6 (m, 4H), 2.8 (m, 1H), 2.42 (m, 1H), 1.98 (m, 3H), 0.7-0.6 (m, 4H).

### Example 23

### 2-{3′-[(Cyclopropanecarbonyl-amino)-methyl]-biphenyl-4-yl}-pyrrolidine-1-carboxylic acid benzyl ester

### Compound 4023

### Cyclopropanecarboxylic acid 3-bromo-benzylamide

3-Bromo-benzylamine hydrochloride (437.3 mg, 2.0 mmol) was dissolved in dichloromethane (15 mL), and the solution was cooled to 0° C. DIPEA (688.0 μL, 3.9 mmol) was then added, and the reaction was stirred at 0° C. for 5 minutes. Then cyclopropanecarbonyl chloride (180.0 μL, 2.3 mmol) was added, and the reaction was stirred at 0° C. for 20 minutes. The reaction was quenched with distilled water, and the solvents were removed. The resulting mixture was redissolved in DMF (10 mL) and purified by reverse phase HPLC to give the desired product.

### Cyclopropanecarboxylic acid 3-(4,4,5,5-tetramethyl-[1,3,2]dioxaborolan-2-yl)-benzylamide

A solution of 4,4,5,5,4′,4′,5′,5′-octamethyl-[2,2′]bi[[1,3,2]dioxaborolanyl] (3.7925 g, 14.93 mmol), cyclopropanecarboxylic acid 3-bromo-benzylamide (1.2595 g, 4.96 mmol), potassium acetate (1.4632 g, 14.91 mmol), and Pd(PPh₃)₂Cl₂ (0.3452 g, 9.9 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-{3′-[(Cyclopropanecarbonyl-amino)-methyl]-biphenyl-4-yl}-pyrrolidine-1-carboxylic acid benzyl ester

### Compound 4023

A solution of cyclopropanecarboxylic acid 3-(4,4,5,5-tetramethyl-[1,3,2]dioxaborolan-2-yl)-benzylamide (66.4 mg, 0.22 mmol), 2-(4-bromo-phenyl)-pyrrolidine-1-carboxylic acid benzyl ester (83.1 mg, 0.23 mmol), and Pd[P(Ph)₃]₄ (12.7 mg, 4.8 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 30.0 mg. MS: 455.2 (M+H⁺); H¹ NMR (DMSO-d₆): δ(ppm) 0.60-0.79 (m, 4H), 1.56-1.96 (m, 4H), 2.20-2.47 (m, 1H), 3.47-3.75 (m, 2H), 4.29-4.40 (s, 2H), 4.80-5.12 (m, 3H), 6.79-6.90 (m, 1H), 7.05-7.61 (m, 12H), 8.53-8.67 (m, 1H).

### Example 24

### (S)-2-(4′-Cyclopropylcarbamoylmethyl-biphenyl-3-ylethenyl)-pyrrolidine-1-carboxylic acid benzyl ester

### Compound 4024

A solution of(S)-2-(3-boronic acid-phenylethynyl)-pyrrolidine-1-carboxylic acid benzyl ester (62.5 mg, 0.18 mmol), 2-(4-bromo-phenyl)-N-cyclopropyl-acetamide (Example 2, 45.8 mg, 0.18 mmol), and Pd[P(Ph)₃]₄ (11.1 mg, 5.4 mol %) in methanol (2 mL), NaHCO₃ (sat. aq., 300 μL), and DMF (1.6 mL) was degassed and heated to 70° C. overnight in a sealed vial. The reaction was cooled, filtered, and purified by reverse phase HPLC to give the desired product. Yield 23.4 mg. MS: 479.6 (M+H⁺); H¹ NMR (DMSO-d₆): δ(ppm) 0.36-0.44 (m, 2H), 0.57-0.66 (m, 2H), 1.86-2.30 (m, 4H), 2.54-2.65 (m, 1H), 4.72-4.84 (m, 1H), 4.98-5.31 (m, 2H), 7.14-7.68 (m, 13H), 8.11-8.17 (m, 1H).

### Example 25

### (S)-2-(6-Pyridin-2-yl-1H-benzoimidazol-2-yl)-pyrrolidine-1-carboxylic acid benzyl ester

### Compound 4025

From 1 eq. of 2-bromo-pyridine following General Procedure A. Yield 41 mg. MS: 399.5 (M+H⁺); H¹-NMR (DMSO-d₆): δ(ppm) 8.7 (m, 1H), 8.4-7.8 (m, 3H), 7.47 (s, 1H), 7.3 (m, 3H), 7.0-6.8 (m, 3H), 5.2-4.8 (m, 3H), 3.70 (m, 1H), 3.55 (m, 1H), 2.02 (m, 4H).

### Example 26

### 2-[4′-(Cyclopropanecarbonyl-amino)-biphenyl-4-yl]-pyrrolidine-1-carboxylic acid benzyl ester

### Compound 4026

### Cyclopropanecarboxylic acid [4-(4,4,5,5-tetramethyl-[1,3,2]dioxaborolan-2-yl)-phenyl]-amide

A solution of 4,4,5,5,4′,4′,5′,5′-octamethyl-[2,2′]bi[[1,3,2]dioxaborolanyl] (3.9704 g, 15.64 mmol), cyclopropanecarboxylic acid (4-bromo-phenyl)-amide (Example 58, 1.2498 g, 5.21 mmol), potassium acetate (1.5272 g, 15.56 mmol), and Pd(PPh₃)₂Cl₂ (0.3639 g, 10.0 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-[4′-(Cyclopropanecarbonyl-amino)-biphenyl-4-yl]-pyrrolidine-1-carboxylic acid benzyl ester

### Compound 4026

A solution of cyclopropanecarboxylic acid [4-(4,4,5,5-tetramethyl-[1,3,2]dioxaborolan-2-yl)-phenyl]-amide (66.2 mg, 0.23 mmol), 2-(4-bromo-phenyl)-pyrrolidine-1-carboxylic acid benzyl ester (82.0 mg, 0.23 mmol), and Pd[P(Ph)₃]₄ (12.3 mg, 4.6 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 22.3 mg. MS: 441.2 (M+H⁺); H¹ NMR (DMSO-d₆): δ(ppm) 0.75-0.86 (m, 4H), 1.68-1.94 (m, 4H), 2.21-2.42 (m, 1H), 3.48-3.73 (m, 2H), 4.80-5.12 (m, 3H), 6.81-6.89 (m, 1H), 7.06-7.40 (m, 6H), 7.50-7.72 (m, 6H), 10.24-10.30 (s, 1H).

### Example 27

### 2-(3′-Cyclopropylcarbamoyl-biphenyl-4-yl)-pyrrolidine-1-carboxylic acid benzyl ester

### Compound 4027

### N-Cyclopropyl-3-(4,4,5,5-tetramethyl-[1,3,2]dioxaborolan-2-yl)-benzamide

A solution of 4,4,5,5,4′,4′,5′,5′-octamethyl-[2,2′]bi[[1,3,2]dioxaborolanyl] (3.9706 g, 15.64 mmol), 3-bromo-N-cyclopropyl-benzamide (Example 1, 1.2463 g, 5.19 mmol), potassium acetate (1.5394 g, 15.69 mmol), and Pd(PPh₃)₂Cl₂ (0.3645 g, 10.0 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-(3′-Cyclopropylcarbamoyl-biphenyl-4-yl)-pyrrolidine-1-carboxylic acid benzyl ester

### Compound 4027

A solution of N-cyclopropyl-3-(4,4,5,5-tetramethyl-[1,3,2]dioxaborolan-2-yl)-benzamide (65.0 mg, 0.23 mmol), 2-(4-bromo-phenyl)-pyrrolidine-1-carboxylic acid benzyl ester (82.6 mg, 0.23 mmol), and Pd[P(Ph)₃]₄ (12.7 mg, 4.9 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 20.5 mg. MS: 441.2 (M+H⁺); H¹ NMR (DMSO-d₆): δ(ppm) 0.54-0.77 (m, 4H), 1.69-1.93 (m, 3H), 2.22-2.45 (m, 1H), 2.80-2.91 (m, 1H), 3.48-3.74 (m, 2H), 4.82-5.14 (m, 3H), 6.81-6.95 (m, 1H), 7.05-7.85 (m, 11H), 7.98-8.08 (m, 1H), 8.47-8.56 (m, 1H).

### Example 28

### (S)-2-[6-(4-Nitro-phenyl)-1H-benzoimidazol-2-yl]-pyrrolidine-1-carboxylic acid benzyl ester

### Compound 4028

From 1 eq of 4-bromo nitrobenzene following General Procedure A. Yield 17 mg. MS: 443.5 (M+H⁺); H¹-NMR (DMSO-d₆): δ(ppm) 8.3 (m, 1H), 8.0-7.7 (m, 5H), 7.3 (m, 3H), 7.0-6.8 (m, 3H), 5.2-4.8 (m, 3H), 3.70 (m, 1H), 3.55 (m, 1H), 2.02 (m, 4H).

### Example 29

### (S)-2-(6-Thiophen-2-yl-1H-benzoimidazol-2-yl)-pyrrolidine-1-carboxylic acid benzyl ester

### Compound 4029

From 1 eq. of 2-bromo-thiophene following General Procedure A. Yield 30 mg. MS: 404.5 (M+H⁺); H¹-NMR (DMSO-d₆): δ(ppm) 7.9-7.7 (m, 4H), 7.6 (m, 2H), 7.3 (m, 2H), 7.1 (m, 1H), 7.0-6.8 (m, 3H), 5.2-4.8 (m, 3H), 3.70 (m, 1H), 3.55 (m, 1H), 2.02 (m, 4H).

### Example 30

### (S)-2-(6-Furan-2-yl-1H-benzoimidazol-2-yl)-pyrrolidine-1-carboxylic acid benzyl ester

### Compound 4030

From 1 eq. of furan-2-yl-lboronic acid following General Procedure B. Yield 5.4 mg. MS: 388.4 (M+H⁺); H¹-NMR (DMSO-d₆): δ(ppm) 7.9-7.7 (m, 4H), 7.3 (m, 3H), 7.1 (m, 2H), 7.0-6.8 (m, 3H), 6.6 (m, 1H), 5.2-4.8 (m, 3H), 3.70 (m, 1H), 3.55 (m, 1H), 2.02 (m, 4H).

### Example 31

### (S)-2-[6-(3-Cyano-phenyl)-1H-benzoimidazol-2-yl]-pyrrolidine-1-carboxylic acid benzyl ester

### Compound 4031

From 1 eq. of benzonitrile-3-boronic acid following General Procedure B. Yield 15 mg. MS: 423.5 (M+H⁺); H¹-NMR (DMSO-d₆): δ(ppm) 8.02-7.8 (m, 8H), 7.37 (m, 3H), 7.0-6.8 (m, 2H), 5.2-4.8 (m, 3H), 3.70 (m, 1H), 3.55 (m, 1H), 2.02 (m, 4H).

### Example 32

### (S)-2-(6-Benzo[1,3]dioxol-5-yl-1H-benzoimidazol-2-yl)-pyrrolidine-1-carboxylic acid benzyl ester

### Compound 4032

From 1 eq. of benzo[1,3]dioxol-5-yl-boronic acid following General Procedure B. Yield 15 mg. MS: 442.5 (M+H⁺); H¹-NMR (DMSO-d₆): δ(ppm) 7.8-7.7 (m, 3H), 7.4-6.8 (m, 8H), 6.07 (s, 2H) 5.2-4.8 (m, 3H), 3.70 (m, 1H), 3.55 (m, 1H), 2.02 (m, 4H).

### Example 33

### (S)-2-[6-(7-Nitro-1H-indol-5-yl)-1H-benzoimidazol-2-yl]-pyrrolidine-1-carboxylic acid benzyl ester

### Compound 4033

From 1 eq. of 5-bromo-7-nitro-1H-indole following General Procedure A. Yield 22 mg. MS: 482.5 (M+H⁺); H¹-NMR (DMSO-d₆): δ(ppm) 8.43 (s, 1H), 8.35 (s, 1H), 8.0-7.7 (m, 4H), 7.6 (m, 1H), 7.3 (m, 3H), 7.0-6.8 (m, 3H), 5.2-4.8 (m, 3H), 3.70 (m, 1H), 3.55 (m, 1H), 2.02 (m, 4H).

### Example 34

### (S)-2-[6-(4-Ureido-phenyl)-1H-benzoimidazol-2-yl]-pyrrolidine-1-carboxylic acid benzyl ester

### Compound 4034

From 1 eq. of (4-bromo-phenyl)-urea following General Procedure A. Yield 48 mg. MS: 456.5 (M+H⁺); H¹-NMR (DMSO-d₆): δ(ppm) 7.9-7.3 (m, 11H), 7.0-6.8 (m, 3H), 6.4 (br s, 1H), 6.1 (1H, br s), 5.2-4.8 (m, 3H), 3.81 (s, 3H), 3.70 (m, 1H), 3.55 (m, 1H), 2.02 (m, 4H).

### Example 35

### (S)-2-[6-(1H-Indol-5-yl)-1H-benzoimidazol-2-yl]-pyrrolidine-1-carboxylic acid benzyl ester

### Compound 4035

From 1 eq. of 1H-indole 5-boronic acid following General Procedure A. Yield 30 mg. MS: 437.5 (M+H⁺); H¹-NMR (DMSO-d₆): δ(ppm) 7.9-7.7 (m, 5H), 7.3 (m, 5H), 7.0-6.8 (m, 2H), 6.5 (m, 1H), 5.2-4.8 (m, 3H), 3.76 (m, 3H), 3.70 (m, 1H), 3.55 (m, 1H), 3.53 (m, 3H), 2.02 (m, 4H).

### Example 36

### (S)-2-[6-(4-Methanesulfonyl-phenyl)-1H-benzoimidazol-2-yl]-pyrrolidine-1-carboxylic acid benzyl ester

### Compound 4036

From 1 eq. of 4-methanesulfonyl-phenyl-boronic acid following General Procedure B. Yield 39 mg. MS: 476.6 (M+H⁺); H¹-NMR (DMSO-d₆): δ(ppm) 8.0 (m, 5H), 7.8 (m, 2H), 7.3 (m, 3H), 7.0-6.8 (m, 2H), 5.2-4.8 (m, 3H) 3.70 (m, 1H), 3.55 (m, 1H), 3.28 (s, 3H), 2.02 (m, 4H).

### Example 37

### (S)-2-{6-[3-(Cyclopropanecarbonyl-amino)-phenyl]-1H-benzoimidazol-2-yl}-pyrrolidine-1-carboxylic acid benzyl ester

### Compound 4037

### Cyclopropanecarboxylic acid (3-bromo-phenyl)-amide

3-Bromo-phenylamine (0.431 mL, 4.0 mmol) was dissolved in dichloromethane (30 mL), and the solution was cooled to 0° C. DIPEA (1.38 mL, 7.9 mmol) was then added, and the reaction was stirred at 0° C. for 5 minutes. Then cyclopropanecarbonyl chloride (0.315 mL, 4.0 mmol) was added, and the reaction was stirred at 0° C. for 20 minutes. The reaction was quenched with distilled water, and the solvents were removed. The resulting mixture was redissolved in DMF (10 mL) and purified by reverse phase HPLC to give the desired product.

### (S)-2-{6-[3-(Cyclopropanecarbonyl-amino)-phenyl]-1H-benzoimidazol-2-yl}-pyrrolidine-1-carboxylic acid benzyl ester

### Compound 4037

A solution of (S)-2-[5-(4,4,5,5-tetramethyl-[1,3,2]dioxaborolan-2-yl)-1H-benzoimidazol-2-yl]pyrrolidine-1-carboxylic acid benzyl ester (90 mg, 0.20 mmol), cyclopropanecarboxylic acid (3-bromo-phenyl)-amide (48.6 mg, 0.20 mmol), and Pd[P(Ph)₃]₄ (13.5 mg, 5.8 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 15.0 mg. MS: 481.2 (M+H⁺); H¹ NMR (DMSO-d₆): δ(ppm) 0.74-0.90 (m, 4H), 1.72-1.87 (m, 1H), 1.90-2.21 (m, 3H), 3.45-3.78 (m, 2H), 4.79-5.16 (m, 2H), 5.19-5.32 (m, 1H), 6.80-7.12 (m, 2H), 7.35-8.06 (m, 10H), 10.28-10.37 (s, 1H).

### Example 38

### (S)-2-(6-{4-[(Cyclopropanecarbonyl-amino)-methyl]-phenyl}-1H-benzoimidazol-2-yl)-pyrrolidine-1-carboxylic acid benzyl ester

### Compound 4038

### Cyclopropanecarboxylic acid 4-bromo-benzylamide

4-Bromo-benzylamine hydrochloride (367.8 mg, 1.7 mmol) was dissolved in dichloromethane (15 mL), and the solution was cooled to 0° C. DIPEA (380 μL, 2.2 mmol) was then added, and the reaction was stirred at 0° C. for 5 minutes. Then cyclopropanecarbonyl chloride (180 μL, 2.3 mmol) was added, and the reaction was stirred at 0° C. for 20 minutes. The reaction was quenched with distilled water, and the solvents were removed. The resulting mixture was redissolved in DMF (10 mL) and purified by reverse phase HPLC to give the desired product.

### (S)-2-(5-{4-[(Cyclopropanecarbonyl-amino)-methyl]-phenyl}-1H-benzoimidazol-2-yl)-pyrrolidine-1-carboxylic acid benzyl ester

### Compound 4038

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (181.2 mg, 0.41 mmol), cyclopropanecarboxylic acid 4-bromo-benzylamide (103.1 mg, 0.41 mmol), and Pd[P(Ph)₃]₄ (24.2 mg, 5.2 mol %) in methanol (4 mL), NaHCO₃ (sat. aq., 600 μL), and DMF (800 μL) was degassed and heated to 70° C. overnight in a sealed vial. The reaction was cooled, filtered, and purified by reverse phase HPLC to give the desired product. The product was then converted to the HCl salt according to the procedure for GL101520. Yield 15.0 mg. MS: 495.2 (M+H⁺); H¹ NMR (DMSO-d₆): δ(ppm) 0.58-0.80 (m, 4H), 1.52-1.69 (m, 1H), 1.90-2.21 (m, 3H), 3.45-3.65 (m, 1H), 4.20-4.48 (m, 2H), 4.80-5.18 (m, 2H), 5.18-5.31 (m, 1H), 6.81-7.98 (m, 12H), 8.50-8.64 (m, 1H).

### Example 39

### (S)-2-[6-(1H-Indol-5-yl)-benzooxazol-2-yl]-pyrrolidine-1-carboxylic acid benzyl ester

### Compound 4039

From 1 eq. 1H-indole 5-boronic acid following General Procedure C. Yield 13.9 mg. MS: 438.5 (M+H⁺); H¹-NMR (DMSO-d₆): δ(ppm) 11.1 (s, 1H), 7.9-7.6 (m, 6H), 7.5-7.3 (m, 7H), 7.1-6.9 (m, 3H), 6.5 (s, 1H), 5.2-4.8 (m, 3H), 3.6-3.5 (m, 4H), 2.39 (m, 1H), 2.12 (m, 3H).

### Example 40

### 2-{4′-[(Cyclopropanecarbonyl-amino)-methyl]-biphenyl-4-yl}-pyrrolidine-1-carboxylic acid benzyl ester

### Compound 4040

### Cyclopropanecarboxylic acid 4-(4,4,5,5-tetramethyl-[1,3,2]dioxaborolan-2-yl)-benzylamide

A solution of 4,4,5,5,4′,4′,5′,5′-octamethyl-[2,2′]bi[[1,3,2]dioxaborolanyl] (1.4976 g, 5.90 mmol), cyclopropanecarboxylic acid 4-bromo-benzylamide (Example 38, 0.4991 g, 1.96 mmol), potassium acetate (0.5708 g, 5.82 mmol), and Pd(PPh₃)₂Cl₂ (0.1403 g, 10.2 mol %) in DMSO (16 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-{4′-[(Cyclopropanecarbonyl-amino)-methyl]-biphenyl-4-yl}-pyrrolidine-1-carboxylic acid benzyl ester

### Compound 4040

A solution of cyclopropanecarboxylic acid 4-(4,4,5,5-tetramethyl-[1,3,2]dioxaborolan-2-yl)-benzylamide (66.3 mg, 0.22 mmol), 2-(4-bromo-phenyl)-pyrrolidine-1-carboxylic acid benzyl ester (82.9 mg, 0.23 mmol), and Pd[P(Ph)₃]₄ (12.7 mg, 5.0 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 30.4 mg. MS: 455.2 (M+H⁺); H¹ NMR (DMSO-d₆): δ(ppm) 0.59-0.78 (m, 4H), 1.55-1.94 (m, 4H), 2.20-2.43 (m, 1H), 3.48-3.75 (m, 2H), 4.23-4.36 (m, 2H), 4.78-5.12 (m, 3H), 6.78-6.89 (m, 1H), 7.00-7.65 (m, 12H), 8.51-8.66 (m, 1H).

### Example 41

### (S)-2-{4′-[(Cyclopropanecarbonyl-amino)-methyl]-biphenyl-3-ylethynyl}-pyrrolidine-1-carboxylic acid benzyl ester

### Compound 4041

A solution of(S)-2-(3-boronic acid-phenylethynyl)-pyrrolidine-1-carboxylic acid benzyl ester (62.5 mg, 0.18 mmol), cyclopropanecarboxylic acid 4-bromo-benzylamide (Example 38, 45.6 mg, 0.18 mmol), and Pd[P(Ph)₃]₄ (11.2 mg, 5.4 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 11.6 mg. MS: 479.5 (M+H⁺); H¹ NMR (DMSO-d₆): δ(ppm) 0.64-0.75 (m, 4H), 1.56-1.67 (m, 1H), 1.86-2.30 (m, 4H), 4.27-4.34 (d, 2H), 4.71-4.84 (m, 1H), 4.96-5.31 (m, 2H), 7.08-7.67 (m, 13H), 8.52-8.62 (m, 1H).

### Example 42

### (S)-2-(6-Pyridin-4-yl-1H-benzoimidazol-2-yl)-pyrrolidine-1-carboxylic acid benzyl ester

### Compound 4042

From 1 eq. of 4-bromo-pyridine following General Procedure A. Yield 24 mg. MS: 399.5 (M+H⁺); M+H⁺); H¹-NMR (DMSO-d₆): δ(ppm) 8.2-8.4 (m, 4H), 7.9 (m, 1H), 7.7 (m, 1H), 7.75 (s, 1H), 7.3 (m, 3H), 7.0-6.8 (m, 3H), 5.2-4.8 (m, 3H), 3.81 (s, 3H), 3.70 (m, 1H), 3.55 (m, 1H), 2.02 (m, 4H).

### Example 43

### 2-(3′-Cyclopropylcarbamoylmethyl-biphenyl-4-yl)-pyrrolidine-1-carboxylic acid benzyl ester

### Compound 4043

### N-Cyclopropyl-2-[3-(4,4,5,5-tetramethyl-[1,3,2]dioxaborolan-2-yl)-phenyl]-acetamide

A solution of 4,4,5,5,4′,4′,5′,5′-octamethyl-[2,2′]bi[[1,3,2]dioxaborolanyl] (3.8025 g, 14.97 mmol), 2-(3-bromo-phenyl)-N-cyclopropyl-acetamide (Example 3, 0.8089 g, 3.18 mmol), potassium acetate (1.4564 g, 14.84 mmol), and Pd(PPh₃)₂Cl₂ (0.3490 g, 15.6 mol %) in DMSO (30 mL) was degassed and heated to 80° C. overnight in a sealed vial. The reaction was cooled, and distilled water and brine were added. The mixture was centrifuged, and the liquid was decanted. The resulting solid was purified by silica gel chromatography to give the desired product.

### 2-(3′-Cyclopropylcarbamoylmethyl-biphenyl-4-yl)-pyrrolidine-1-carboxylic acid benzyl ester

### Compound 4043

A solution of N-cyclopropyl-2-[3-(4,4,5,5-tetramethyl-[1,3,2]dioxaborolan-2-yl)-phenyl]-acetamide (66.1 mg, 0.22 mmol), 2-(4-bromo-phenyl)-pyrrolidine-1-carboxylic acid benzyl ester (81.8 mg, 0.23 mmol), and Pd[P(Ph)₃]₄ (12.8 mg, 5.0 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 37.8 mg. MS: 455.2 (M+H⁺); H¹ NMR (DMSO-d₆): δ(ppm) 0.35-0.44 (m, 2H), 0.55-0.70 (m, 2H), 1.66-1.92 (m, 3H), 2.20-2.45 (m, 1H), 2.53-2.65 (m, 1H), 3.35-3.44 (s, 2H), 3.46-3.72 (m, 2H), 5.30-5.62 (m, 3H), 6.78-6.88 (m, 1H), 7.03-7.57 (m, 12H), 8.11-8.20 (m, 1H).

### Example 44

### 2-(4′-Cyclopropylcarbamoyl-biphenyl-4-yl)-pyrrolidine-1-carboxylic acid benzyl ester

### Compound 4044

A solution of N-cyclopropyl-4-(4,4,5,5-tetramethyl-[1,3,2]dioxaborolan-2-yl)-benzamide (Example 10, 64.5 mg, 0.22 mmol), 2-(4-bromo-phenyl)-pyrrolidine-1-carboxylic acid benzyl ester (82.7 mg, 0.23 mmol), and Pd[P(Ph)₃]₄ (13.1 mg, 4.9 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 39.0 mg. MS: 441.2 (M+H⁺); H¹ NMR (DMSO-d₆): δ(ppm) 0.540.75 (m, 4H), 1.69-1.92 (m, 3H), 2.23-2.43 (m, 1H), 2.79-2.90 (m, 1H), 4.82-5.11 (m, 3H), 6.80-6.89 (m, 1H), 7.04-7.41 (m, 6H), 7.57-7.95 (m, 6H), 8.42-8.50 (m, 1H).

### Example 45

### (S)-2-[6-(3-Acetyl-phenyl)-1H-benzoimidazol-2-yl]-pyrrolidine-1-carboxylic acid benzyl ester

### Compound 4045

From 1 eq. of 1-(3-bromo-phenyl)-ethanone following General Procedure A. Yield 21 mg. MS: 440.5 (M+H⁺); H¹-NMR (DMSO-d₆): δ(ppm) 8.1 (m, 1H), 7.9 (m, 2H), 7.7 (m, 3H), 7.65 (m, 1H), 7.3 (m, 3H), 7.0-6.8 (m, 3H), 5.2-4.8 (m, 3H), 3.70 (m, 1H), 3.55 (m, 1H), 2.66 (s, 3H), 2.02 (m, 4H).

### Example 46

### (S)-2-(6-Thiophen-3-yl-1H-benzoimidazol-2-yl)-pyrrolidine-1-carboxylic acid benzyl ester

### Compound 4046

From 1 eq. of 3-bromo-thiophene following General Procedure A. Yield 10 mg. MS: 404.5 (M+H⁺); H¹-NMR (DMSO-d₆): δ(ppm) 8.2-8.4 (m, 4H), 7.9 (m, 1H), 7.7 (m, 1H), 8.0-7.6 (m, 6H), 7.3 (m, 3H), 7.0-6.8 (m, 3H), 5.2-4.8 (m, 3H), 3.70 (m, 1H), 3.55 (m, 1H), 2.02 (m, 4H).

### Example 47

### (S)-2-[6-(9H-Carbazol-3-yl)-1H-benzoimidazol-2-yl]-pyrrolidine-1-carboxylic acid benzyl ester

### Compound 4047

From 1 eq. of 3-bromo-9H-carbazole following General Procedure A. Yield 18 mg. MS: 489.5 (M-NHCOCH₃); H¹-NMR (DMSO-d₆): δ(ppm) 8.5 (s, 1H), 8.2 (m, 1H), 8.0-7.7 (m, 6H), 7.6 (m, 1H), 7.5 (m, 1H), 7.3 (m, 3H), 7.1-6.8 (m, 4H), 5.2-4.8 (m, 3H), 3.70 (m, 1H), 3.55 (m, 1H), 2.02 (m, 4H).

### Example 48

### (S)-2-[6-(4-Cyclopropylcarbamoyl-phenyl)-1H-imidazo[4,5-b]pyridin-2-yl]-pyrrolidine-1-carboxylic acid benzyl ester

### Compound 4048

### (S)-2-(3-Amino-5-bromo-pyridin-2-ylcarbamoyl)-pyrrolidine-1-carboxylic acid benzyl ester

Z-protected (S)-proline (500 mg) was dissolved in DMF (15 mL) and treated with HATU (1.1 eq. 800 mg) and DIPEA (2.1 eq, 0.78 mL) and stirred for 15 minutes. 5-Bromo-pyridine-2,3-diamine (1 eq, 376 mg) was added and the mixture stirred at ambient temperature overnight. The reaction was cooled, filtered and the solvents removed. The resulting mixture was redissolved in 5 mL of 90% DMF, 10% water with 0.1% TFA and purified by reverse phase HPLC to give the product. MS: 419.5 (M+H⁺)

### (S)-2-(6-Bromo-1H-imidazo[4,5-b]pyridin-2-yl)-pyrrolidine-1-carboxylic acid benzyl ester

(S)-2-(3-Amino-5-bromo-pyridin-2-ylcarbamoyl)-pyrrolidine-1-carboxylic acid benzyl ester was dissolved in HOAc (50 mL) and heated to 110 deg. C. for 12 hours. The reaction was cooled, filtered and the solvents removed. The resulting mixture was redissolved in 5 mL of 90% DMF, 10% water with 0.1% TFA and purified by reverse phase HPLC to give the product. MS: 401.5 (M+H⁺)

### (S)-2-[6-(4-Cyclopropylcarbamoyl-phenyl)-1H-imidazo[4,5-b]pyridin-2-yl]-pyrrolidine-1-carboxylic acid benzyl ester

### Compound 4048

A solution of (S)-2-(6-Bromo-1H-imidazo[4,5-b]pyridin-2-yl)-pyrrolidine-1-carboxylic acid benzyl ester (80 mg, 0.2 mmol), N-cyclopropyl-4-(4,4,5,5-tetramethyl-[1,3,2]dioxaborolan-2-yl)-benzamide (Example 10, 1 eq), Pd[P(Ph)₃]₄ (5 mol %, 11 mg), in methanol (2 mL), NaHCO₃ (sat. aq., 300 μL) and DMF (400 μL) was degassed and heated to 70° C. overnight in a sealed vial. The reaction was cooled, filtered and the solvents removed. The resulting mixture was redissolved in 5 mL of 90% DMF, 10% water with 0.1% TFA and purified by reverse phase HPLC to give the products. Yield 8.2 mg. MS: 482.5 (M+H⁺); H¹-NMR (DMSO-d₆): δ(ppm) 8.7 (m, 1H), 8.5 (m, 1H), 8.3 (m, 1H), 7.9-7.8 (m, 5H), 7.3 (m, 2H), 6.9-6.8 (m, 2H), 5.2-4.8 (m, 3H), 3.8 (m, 1H), 3.5 (m, 1H), 2.8 (m, 1H), 2.42 (m, 1H), 2.02 (m, 2H), 0.7-0.6 (m, 4H)

### Example 49

### (S)-2-[6-(4-Cyano-phenyl)-1H-benzoimidazol-2-yl]-pyrrolidine-1-carboxylic acid benzyl ester

### Compound 4049

From 1 eq. of benzonitrile-4-boronic acid following General Procedure B. Yield 27 mg. MS: 423.5 (M+H⁺); H¹-NMR (DMSO-d₆): δ(ppm)) 8.02-7.8 (m, 8H), 7.37 (m, 3H), 7.0-6.8 (m, 2H), 5.2-4.8 (m, 3H), 3.70 (m, 1H), 3.55 (m, 1H), 2.02 (m, 4H).

### Example 50

### (S)-2-[6-(2,4-Dimethoxy-pyrimidin-5-yl)-1H-benzoimidazol-2-yl]-pyrrolidine-1-carboxylic acid benzyl ester

### Compound 4050

From 1 eq. of 2,4-dimethoxy-pyrimidine 5-boronic acid following General Procedure B. Yield 11 mg. MS: 460.5 (M+H⁺); H¹-NMR (DMSO-d₆): δ(ppm) 8.5 (s, 1H), 7.9-7.5 (m, 5H), 7.3 (m, 5H), 7.0-6.8 (m, 2H), 6.5 (m, 1H), 5.2-4.8 (m, 3H), 3.76 (m, 3H), 3.70 (m, 1H), 3.55 (m, 1H), 3.53 (m, 3H), 2.02 (m, 4H).

### Example 51

### (S)-2-[6-(2-Methyl-benzothiazol-5-yl)-1H-benzoimidazol-2-yl]-pyrrolidine-1-carboxylic acid benzyl ester

### compound 4051

From 1 eq. of 5-Bromo-2-methyl-benzothiazole following General Procedure A. Yield 38 mg. MS: 469.6 (M+H⁺); H¹-NMR (DMSO-d₆): δ(ppm) 8.2-7.7 (m, 7H), 7.3 (m, 2H), 7.0-6.8 (m, 3H), 5.2-4.8 (m, 3H), 3.70 (m, 1H), 3.55 (m, 1H), 2.83 (s, 3H), 2.02 (m, 4H).

### Example 52

### (S)-2-[6-(2-Hydroxy-phenyl)-1H-benzoimidazol-2-yl]-pyrrolidine-1-carboxylic acid benzyl ester

### Compound 4052

From 1 eq. of 2-(5,5-Dimethyl-[1,3,2]dioxaborinan-2-yl)-phenol following General Procedure B. Yield 39 mg. MS: 414.5 (M+H⁺); H¹-NMR (DMSO-d₆): δ(ppm) 7.9-7.6 (m, 4H), 7.4-6.8 (m, 8H), 5.2-4.8 (m, 3H), 3.70 (m, 1H), 3.55 (m, 1H), 2.02 (m, 4H).

### Example 53

### (S)-2-[6-(3-Amino-phenyl)-1H-benzoimidazol-2-yl]-pyrrolidine-1-carboxylic acid benzyl ester

### Compound 4053

From 1 eq. of 3-aminophenylboronic acid following General Procedure B. Yield 31 mg. MS: 413.5 (M+H⁺); H¹-NMR (DMSO-d₆): δ(ppm) 7.8-7.7 (m, 3H), 7.4 (m, 4H), 7.2 (m, 2H), 7.0-6.8 (m, 3H), 5.2-4.8 (m, 3H), 3.70 (m, 1H), 3.55 (m, 1H), 2.02 (m, 4H).

### Example 54

### (S)-2-[6-(3-Hydroxy-phenyl)-1H-benzoimidazol-2-yl]-pyrrolidine-1-carboxylic acid benzyl ester

### Compound 4054

From 1 eq. of 3-bromo-phenol following General Procedure A. Yield 22 mg. MS: 414.5 (M+H⁺); H¹-NMR (DMSO-d₆): δ(ppm) 7.8-7.7 (m, 5H), 7.3 (m, 3H), 7.0-6.8 (m, 4H), 5.2-4.8 (m, 3H), 3.70 (m, 1H), 3.55 (m, 1H), 2.02 (m, 4H).

### Example 55

### (S)-2-{[4-(4-Cyclopropylcarbamoyl-phenyl)-thiazol-2-ylamino]-methyl}-pyrrolidine-1-carboxylic acid benzyl ester

### Compound 4055

### 4-(2-Amino-thiazol-4-yl)-benzoic acid

A solution of 4-acetyl-benzoic acid (10 g, 61 mmol), in HOAc (400 mL) at 55° C. was treated with bromine (1 eq., 3.12 mL) dropwise over 10 minutes. After 90 minutes the reaction was cooled, the acetic acid was removed, ethyl acetate (50 mL) was added and then removed to get rid of the remainder of the acetic acid. The crude bromo ketone was then dissolved in ethanol (200 mL) with NaOAc (12 g) and thiourea (1 eq. 4.4 g) added. The suspension was stirred at room temperature for 15 hours. The solvents were removed and the solids washed with water (3×100 mL) then ether:ethanol (4:1, 3×100 mL) and dried to give the product as a tan solid. MS: 221.2 (M+H⁺).

### 4-(2-Amino-thiazol-4-yl)-N-cyclopropyl-benzamide

4-(2-Amino-thiazol-4-yl)-benzoic acid (4.4 g, 20 mmol) was dissolved in DMF (100 mL) and treated with HATU (1.1 eq. 8.4 g) and DIPEA (2.1 eq, 7.5 mL) and stirred for 15 minutes. Then cyclopropyl amine (1.1 eq, 1.5 mL) was added and the mixture stirred at ambient temperature for 1 hour. The reaction was diluted with 100 mL of water, extracted with EtOAc (3×100 mL) dried with brine (100 mL) and then Na₂SO₄ and the solvents removed. The resulting solid was triturated with ether to give the product >95% purity by HPLC. MS: 260.3 (M+H).

### (S)-2-{[4-(4-Cyclopropylcarbamoyl-phenyl)-thiazol-2-ylamino]-methyl}-pyrrolidine-1-carboxylic acid benzyl ester

### Compound 4055

A solution of 4-(2-Amino-thiazol-4-yl)-N-cyclopropyl-benzamide (259 mg, 1 mmol), (S)-2-Formyl-pyrrolidine-1-carboxylic acid benzyl ester (1 eq., 233 mg) in methanol (1 mL) was treated with NaBH₃CN (2 eq. 211 mg) and heated to 50° C. overnight in a sealed vial. Another portion of NaBH₃CN (100 mg) was added and the mixture heated for another 1 hour. The reaction was cooled, filtered and the solvents removed. The resulting mixture was redissolved in 5 mL of 90% DMF, 10% water with 0.1% TFA and purified by reverse phase HPLC to give the product. Yield 20 mg. MS: 476.3 (M+H). H¹-NMR (DMSO-d₆): δ(ppm) 8.3 (s, 1H), 7.8 (m, 5H), 7.3-7.1 (m, 8H), 5.1 (m, 4H), 3.5 (m, 2H), 3.3 (m, 2H), 1.9 (m, 4H), 0.7-0.5 (m, 4H).

### Example 56

### (S)-2-[5-(4-Cyclopropylcarbamoyl-thiazol-2-yl)-1H-benzoimidazol-2-yl]-pyrrolidine-1-carboxylic acid benzyl ester

### Compound 4056

From 1 eq of 2-bromo-thiazole-4-carboxylic acid cyclopropylamide following General Procedure A. Yield 11.6 mg. MS: 488.5 (M+H⁺); H¹-NMR (DMSO-d₆): δ(ppm) 8.5 (m, 1H), 8.2 (m, 2H), 8.1 (m, 1H), 7.8 (m, 1H), 7.3 (m, 3H), 7.0-6.8 (m, 2H), 5.2-4.8 (m, 3H), 3.70 (m, 1H), 3.55 (m, 1H), 2.90 (m, 1H), 2.02 (m, 4H), 0.71 (m, 4H).

### Example 57

### (S)-2-(6-{3-[(Cyclopropanecarbonyl-amino)-methyl]-phenyl}-1H-benzoimidazol-2-yl)-pyrrolidine-1-carboxylic acid benzyl ester

### Compound 4057

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (90.6 mg, 0.20 mmol), cyclopropanecarboxylic acid 3-bromo-benzylamide (Example 23, 51.8 mg, 0.20 mmol), and Pd[P(Ph)₃]₄ (13.5 mg, 5.8 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 16.4 mg. MS: 495.2 (M+H⁺); H¹ NMR (DMSO-d₆): δ(ppm) 0.56-0.72 (m, 4H), 1.51-1.66 (m, 1H), 1.89-2.20 (m, 3H), 4.20-4.39 (m, 2H), 4.79-5.16 (m, 2H), 5.18-5.32 (m, 1H), 6.76-7.98 (m, 12H), 8.51-8.66 (m, 1H).

### Example 58

### (S)-2-{6-[4-(Cyclopropanecarbonyl-amino)-phenyl]-1H-benzoimidazol-2-yl}-pyrrolidine-1-carboxylic acid benzyl ester

### Compound 4058

### Cyclopropanecarboxylic acid (4-bromo-phenyl)-amide

4-Bromo-phenylamine (690 mg, 4.0 mmol) was dissolved in dichloromethane (30 mL), and the solution was cooled to 0° C. DIPEA (1.38 mL, 7.9 mmol) was then added, and the reaction was stirred at 0° C. for 5 minutes. Then cyclopropanecarbonyl chloride (0.315 mL, 4.0 mmol) was added, and the reaction was stirred at 0° C. for 20 minutes. The reaction was quenched with distilled water, and the solvents were removed. The resulting mixture was redissolved in DMF (10 mL) and purified by reverse phase HPLC to give the desired product.

### (S)-2-{6-[4-(Cyclopropanecarbonyl-amino)-phenyl]-1H-benzoimidazol-2-yl}-pyrrolidine-1-carboxylic acid benzyl ester

### Compound 4058

A solution of (S)-2-[5-(4,4,5,5-tetramethyl-[1,3,2]dioxaborolan-2-yl)-1H-benzoimidazol-2-yl]-pyrrolidine-1-carboxylic acid benzyl ester (90 mg, 0.20 mmol), cyclopropanecarboxylic acid (4-bromo-phenyl)-amide (49.0 mg, 0.20 mmol), and Pd[P(Ph)₃]₄ (13.0 mg, 5.6 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 15.0 mg. MS: 481.2 (M+H⁺); H¹ NMR (DMSO-d₆): δ(ppm) 0.75-0.87 (m, 4H), 1.74-1.85 (m, 1H), 1.92-2.21 (m, 3H), 3.65-3.78 (m, 2H), 4.80-5.16 (m, 2H), 5.19-5.32 (m, 1H), 6.80-7.15 (m, 2H), 7.23-7.45 (m, 2H), 7.56-7.93 (m, 8H), 10.26-10.37 (s, 1H).

### Example 59

### (S)-2-(6-Bromo-1H-imidazo[4,5-b]pyridin-2-yl)-pyrrolidine-1-carboxylic acid benzyl ester

### Compound 4059

### (S)-2-(3-Amino-5-bromo-pyridin-2-ylcarbamoyl)-pyrrolidine-1-carboxylic acid benzyl ester

Z-protected (S)-proline (500 mg) was dissolved in DMF (15 mL) and treated with HATU (1.1 eq. 800 mg) and DIPEA (2.1 eq, 0.78 mL) and stirred for 15 minutes. Then 5-bromo-pyridine-2,3-diamine (1 eq, 376 mg) was added and the mixture stirred at ambient temperature overnight. The reaction was cooled, filtered and the solvents removed. The resulting mixture was redissolved in 5 mL of 90% DMF, 10% water with 0.1% TFA and purified by reverse phase HPLC to give the product. MS: 419.3 (M+H⁺)

### (S)-2-(6-Bromo-1H-imidazo[4,5-b]pyridin-2-yl)-pyrrolidine-1-carboxylic acid benzyl ester

### Compound 4059

(S)-2-(3-Amino-5-bromo-pyridin-2-ylcarbamoyl)-pyrrolidine-1-carboxylic acid benzyl ester was dissolved in HOAc (10 mL) and heated to 110° C. for 12 hours. The reaction was cooled, filtered and the solvents removed. The resulting mixture was redissolved in 5 mL of 90% DMF, 10% water with 0.1% TFA and purified by reverse phase HPLC to give the product. Yield 6.2 mg. MS: 401.5 (M+H⁺). H¹-NMR (DMSO-d₆): δ(ppm) 8.4 (m, 1H), 8.1 (m, 1H), 7.3 (m, 2H), 7.1-6.7 (m, 3H), 5.2-4.8 (m, 3H), 3.5 (m, 2H), 1.91 (m, 4H).

### Example 60

### (S)-2-(4′-Cyclopropylcarbamoyl-biphenyl-3-ylethynyl)-pyrrolidine-1-carboxylic acid benzyl ester

### Compound 4060

A solution of (S)-2-(3-boronic acid-phenylethynyl)-pyrrolidine-1-carboxylic acid benzyl ester (62.5 mg, 0.18 mmol), 4-bromo-N-cyclopropyl-benzamide (Example 10, 43.3 mg, 0.18 mmol), and Pd[P(Ph)₃]₄ (11.2 mg, 5.4 mol %) in methanol (2 mL), NaHCO₃ (sat. aq., 300 μL), and DMF (400 μL) was degassed and heated to 70° C. overnight in a sealed vial. The reaction was cooled, filtered, and purified by reverse phase HPLC to give the desired product. Yield 10.1 mg. MS: 465.5 (M+H⁺); H¹ NMR (DMSO-d₆): δ(ppm) 0.54-0.70 (m, 4H), 1.86-2.28 (m, 3H), 2.78-2.90 (m, 1H), 4.71-4.82 (m, 2H), 4.96-5.12 (m, 2H), 5.18-5.29 (m, 1H), 7.30-7.90 (m, 13H), 8.41-8.8.49 (m, 1H).

### Biological Examples

### Example 1

### Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles et al. In vitro assays have been reported in Ferrari et al. J. of Vir., 73:1649-1654, 1999; Ishii et al., Hepatology, 29:1227-1235, 1999; Lohmann et al, J. of Bio. Chem., 274:10807-10815, 1999; and Yamashita et al., J. of Bio. Chem., 273:15479-15486, 1998.

### Replicon Assay

A cell line, ET (Huh-lucubineo-ET) was used for screening of compounds of the present invention for inhibiting HCV replication. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×10⁴ cells/well in the 96 well plates and incubated for 24 hrs before adding testing compounds. Then the compounds were added to the cells to achieve a final concentration of 0.1 nM to 50 μM and a final DMSO concentration of 0.5%. Luciferase activity were measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication was plotted relative to no compound control. Under the same condition, cytotoxicity of the compounds was determined using cell proliferation reagent, WST-1 (Roche, Germany). The compounds showing antiviral activities, but no significant cytotoxicities were chosen to determine EC₅₀ and TC₅₀. For these determinations, a 10 point 2-fold serial dilution for each compound was used, which spans a concentration range of 1000 fold. EC₅₀ and similarly TC₅₀ values were calculated by fitting % inhibition at each concentration to the following equation:

% inhibition=100%/[(IC50/[I])^{b}+1]

where b is Hill's coefficient.

Preferably, when tested at 100 μM the compounds of this invention will exhibit a % inhibition of at least 30% and more preferably a % inhibition of at least 50%.

When tested at 10 μM, the compounds in Table 1 were found to have the indicated percent inhibition values shown in Table 2. Compounds of Table 1 with % inhibition of less than 1% are not included in Table 2, but may have greater activity when tested at higher concentrations. In some preferred embodiments compounds of Formula I will have a % inhibition of at least 20% when tested at 10 μM. In other embodiments the compounds of Formula I will have a % inhibition of at least 50% when tested at 10 μM.

TABLE 2

| Compound # | % inhibition at 10 μM |
|---|---|
| 4001 | 94.5 |
| 4002 | 33.3 |
| 4003 | 97.0 |
| 4004 | 81.8 |
| 4005 | 1.7 |
| 4006 | 99.8 |
| 4007 | 4.4 |
| 4008 | 13.6 |
| 4009 | 31.7 |
| 4010 | 96.8 |
| 4011 | 87.0 |
| 4014 | 12.7 |
| 4017 | 42.4 |
| 4019 | 50.5 |
| 4020 | 96.1 |
| 4021 | 64.5 |
| 4022 | 14.9 |
| 4024 | 45.9 |
| 4026 | 47.6 |
| 4029 | 78.1 |
| 4030 | 66.8 |
| 4032 | 25.8 |
| 4033 | 97.5 |
| 4034 | 24.1 |
| 4035 | 99.9 |
| 4037 | 97.6 |
| 4038 | 97.0 |
| 4039 | 49.6 |
| 4041 | 45.8 |
| 4043 | 40.0 |
| 4044 | 53.1 |
| 4045 | 99.8 |
| 4047 | 98.2 |
| 4049 | 20.0 |
| 4051 | 32.8 |
| 4054 | 99.3 |
| 4055 | 44.0 |
| 4057 | 99.4 |
| 4058 | 97.9 |
| 4060 | 48.4 |

### Formulation Examples

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration.

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co.) | 1.0 | g |
| flavoring | 0.035 | mL |
| colorings | 0.5 | mg |
| distilled water | q.s. to 100 | mL |

### Example 4

### Injectable Formulation

The following ingredients are mixed to form an injectable formulation.

| Ingredient | Quantity per tablet, mg | |
|---|---|---|
| Compound of the invention | 0.2 mg-20 mg | |
| sodium acetate buffer solution, 0.4 M | 2.0 | mL |
| HCl (1N) or NaOH (1N) | q.s. to suitable pH | |
| water (distilled, sterile) | q.s. to 20 | mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | balance |

What is claimed is:

1. A compound of Formula (I) or a stereoisomer, tautomer, pharmaceutically acceptable salt, or prodrug thereof, wherein: 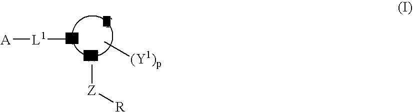
A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
L¹ is a bond, C₁-C₃ alkylene, C₁-C₂ heteroalkylene, C₂-C₃ alkenylene, or C₂-C₃ alkynylene;
T is C₂-C₆ alkylene or C₁-C₅ heteroalkylene and forms a 4-8 membered ring with V and W;
V and W are both CH, or one of V or W is CH and the other of V or W is N;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

2. A compound of claim 1 wherein A is selected from the group consisting of

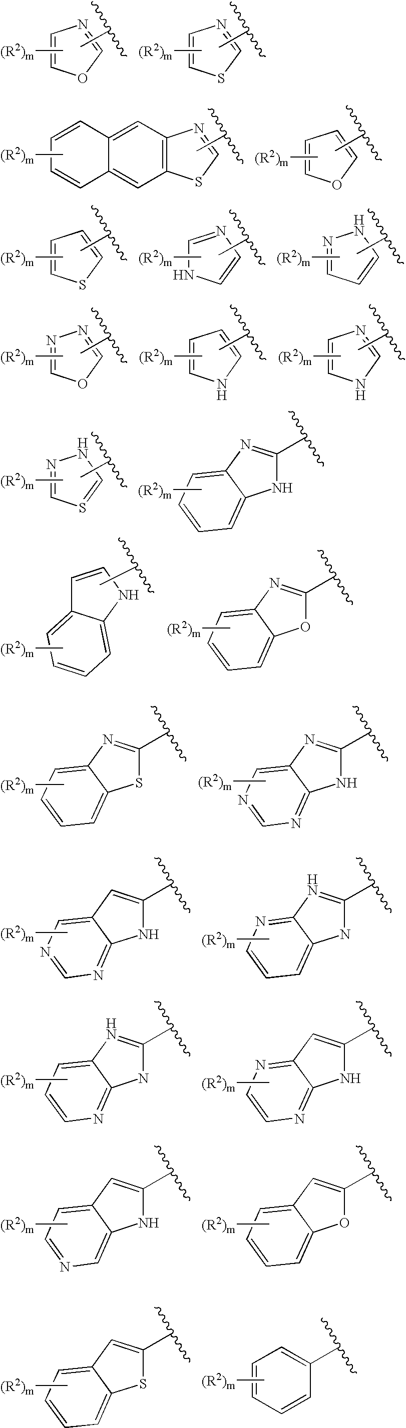

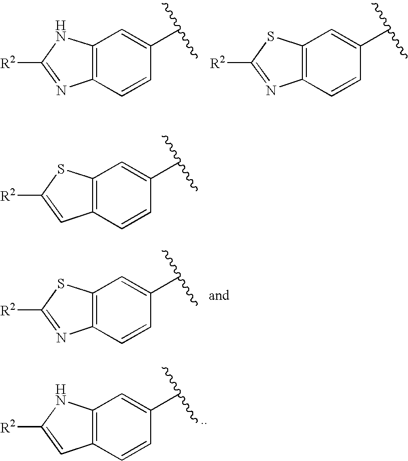

3. A compound of any one of claims 1 to 2 wherein L¹ is a bond.

4. A compound of any one of claims 1 to 2 wherein L¹ is C₂ alkynylene.

5. A compound of claim 1 wherein at least one of R² is R³-L- wherein R³ is selected from the group consisting of aryl, substituted aryl, heteroaryl, substituted heteroaryl, heterocyclic, and substituted heterocyclic; and L, defined in the R³-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R³ is heterocyclic or substituted heterocyclic.

6. A compound of claim 5 wherein R³ is substituted phenyl.

7. A compound of claim 1 wherein V is C and W is N.

8. A compound of claim 1 wherein Z is C(O).

9. A compound of claim 1 wherein R is OCH₂R¹ and R¹ is phenyl or substituted phenyl.

10. A compound of claim 1 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

11. A compound of claim 1 having Formula (II) or a stereoisomer, tautomer, pharmaceutically acceptable salt, or prodrug thereof, wherein: 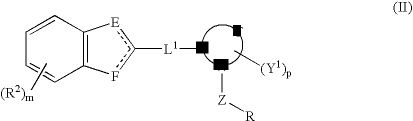
one of E or F is —N═ and the other of E or F is —S—, —O— or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
L¹ is a bond, C₁-C₃ alkylene, C₁-C₂ heteroalkylene, C₂-C₃ alkenylene, or C₂-C₃ alkynylene;
T is C₂-C₆ alkylene or C₁-C₅ heteroalkylene and forms a 4-8 membered ring with V and W;
V and W are both CH, or one of V or W is CH and the other of V or W is N;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

12. A compound of claim 11 wherein L¹ is a bond.

13. A compound of claim 11 wherein L¹ is C₂ alkynylene

14. A compound of claim 11 wherein at least one of R² is R³-L- wherein R³ is selected from the group consisting of aryl, substituted aryl, heteroaryl, substituted heteroaryl, heterocyclic, and substituted heterocyclic; and L, defined in the R³-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R³ is heterocyclic or substituted heterocyclic.

15. A compound of claim 14 wherein R³ is substituted phenyl.

16. A compound of claim 11 wherein V is C and W is N.

17. A compound of claim 11 wherein Z is C(O).

18. A compound of claim 11 wherein R is OCH₂R¹ and R¹ is phenyl or substituted phenyl.

19. A compound of claim 11 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

20. A compound of claim 1 having Formula (III) or a stereoisomer, tautomer, pharmaceutically acceptable salt, or prodrug thereof, wherein: 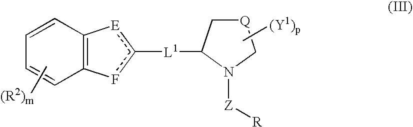
one of E or F is —N═ and the other of E or F is —S—, —O— or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
L¹ is a bond, C₁-C₃ alkylene, C₁-C₂ heteroalkylene, C₂-C₃ alkenylene, or C₂-C₃ alkynylene;
Q is selected from the group consisting of CH₂, CH(Y¹), C(Y¹)(Y¹), S, and O;
p is 0, 1 or 2;
Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, halo, hydroxy, alkoxy, substituted alkoxy, ═CH₂, oxo, or two Y¹ groups together with the atoms to which they are bound form a phenyl, 4-7 membered cycloalkyl, or 4-7 membered heterocyclic ring where the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

21. A compound of claim 20 wherein at least one of R² is R³-L- wherein R³ is selected from the group consisting of aryl, substituted aryl, heteroaryl, substituted heteroaryl, heterocyclic, and substituted heterocyclic; and L, defined in the R³-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R³ is heterocyclic or substituted heterocyclic.

22. A compound of claim 21 wherein R³ is substituted phenyl.

23. A compound of claim 20 wherein Z is C(O).

24. A compound of claim 20 wherein R is OCH₂R¹ and R¹ is phenyl or substituted phenyl.

25. A compound of claim 20 wherein Q is S, CH₂, or O.

26. A compound of claim 20 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

27. A compound of claim 1 or a stereoisomer, tautomer, pharmaceutically acceptable salt, or prodrug thereof selected from Table 1.

28. A pharmaceutical composition comprising a pharmaceutically acceptable carrier and a therapeutically effective amount of a compound, stereoisomer, tautomer, pharmaceutically acceptable salt, or prodrug thereof of claim 1.

29. A method for treating a viral infection in a patient mediated at least in part by a virus in the Flaviviridae family of viruses which method comprises administering to the patient a compound, stereoisomer, tautomer, pharmaceutically acceptable salt, or prodrug thereof of claim 1.

30. The method of claim 29 wherein said viral infection is a hepatitis C mediated viral infection.

31. The method of claim 29 in combination with the administration of a therapeutically effective amount of one or more agents active against hepatitis C virus.

32. The method of claim 31 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

33. The method of claim 31 wherein said agent active against hepatitis C virus is interferon.
